(19) United States
(12) Patent Application Publication
(10) Pub. No.: US 2009/0285878 A1
(43) Pub. Date: Nov. 19, 2009

(54) Compositions and methods for stabilizing liposomal drug formulations

(75) Inventors: Michael J. Hope, Vancouver (CA); Barbara Mui, Vancouver (CA); Thomas D. Madden, Vancouver (CA)

Correspondence Address: LAHIVE & COCKFIELD, LLP;FLOOR 30, SUITE 3000, ONE POST OFFICE SQUARE, BOSTON, MA, 02109, US

(73) Assignee: Tekmira Pharmaceuticals Corporation, Burnaby, BC (CA)

(21) Appl. No.: 11/667,138

(22) PCT Filed: Nov. 4, 2005
(86) PCT No.: PCT/US05/40061

§ 371 Date: Oct. 27, 2008

Related U.S. Application Data
(60) Provisional application No. 60/625,199, filed on Nov. 5, 2004.

(51) Int. Cl. A61K 31/4745; A61K 9/127; A61P 35/00

(52) U.S. Cl. 424/450; 514/283

(57) ABSTRACT

The present invention is directed to liposomal compositions comprising a camptothecin, which are optimized to reduce camptothecin degradation and/or precipitation of camptothecin degradation products in the external medium. The invention further provides improved methods of formulating liposomal camptothecins, kits comprising liposome-encapsulated camptothecins, and methods of using the same to treat a variety of diseases and disorders, including cancer.

### BACKGROUND OF THE INVENTION

1. Field of the Invention

The present invention is directed to novel liposomal camptothecin formulations and kits having increased drug stability.

2. Description of the Related Art

A major challenge facing medical science and the pharmaceutical industry, in particular, is to develop methods for providing camptothecins to appropriate tissues or cells at a sufficient dosage to provide a therapeutic benefit, without prohibitively harming the patient being treated. Accordingly, it is an important goal of the pharmaceutical industry to develop drug delivery methods that provide increased efficacy with decreased associated toxicity. A variety of different general approaches have been taken, with various degrees of success. These include, e.g., the use of implantable drug delivery devices, the attachment of targeting moieties to therapeutic compounds, and the encapsulation of therapeutic compounds, e.g., in liposomes, to alter release rates and toxicity.

Liposomal encapsulation of therapeutic compounds has shown significant promise in controlled drug delivery. For example, some lipid-based formulations provide longer half-lives in vivo, superior tissue targeting, or decreased toxicity. In efforts to develop more effective therapeutic treatments, attempts have been made to encapsulate a variety of therapeutic compounds in liposomes. For example, many anticancer or antineoplastic drugs have been encapsulated in liposomes. These include alkylating agents, nitrosoureas, cisplatin, antimetabolites, vinca alkaloids, camptothecins, taxanes and anthracyclines. Studies with liposomes containing anthracycline antibiotics have clearly shown reduction of cardiotoxicity.

Liposomal formulations of drugs modify drug pharmacokinetics as compared to their free drug counterpart, which is not liposome-encapsulated. For a liposomal drug formulation, drug pharmacokinetics are largely determined by the rate at which the carrier is cleared from the blood and the rate at which the drug is released from the carrier. Considerable efforts have been made to identify liposomal carrier compositions that show slow clearance from the blood, and long-circulating carriers have been described in numerous scientific publications and patents. Efforts have also been made to control drug leakage or release rates from liposomal carriers, using for example, various lipid components or a transmembrane potential to control release.

Camptothecins are anticancer agents based on the natural product camptothecin. Although camptothecin itself has antitumor activity it is highly insoluble in water and consequent difficulties in administration may have contributed to the unpredictable toxicity seen in early clinical studies (Gottlieb et al., 1970, Cancer Chemotherapy Reports 54:461-70; Muggia et al., 1972, Cancer Chemotherapy Reports 56: 515-521). Subsequent studies therefore focused on the development of water-soluble camptothecin derivatives and their clinical evaluation (reviewed in Bailly, 2000, Current Medicinal Chemistry, 7: 39-58; Dallavalle et al., 2001, Journal of Medicinal Chemistry 44: 3264-3274). These water-soluble derivatives include topotecan and irinotecan, which are approved agents for use in the treatment of various cancers. These water-soluble derivatives rely on the addition of charged or polar groups to the camptothecin backbone to increase aqueous solubility. Consequently however, degradation products of these agents, wherein the charged or polar group is modified or lost, are usually highly insoluble and tend to form precipitates (Kearney et al., 1996, International Journal of Pharmaceutics 127: 229-237). Pharmaceutical products intended for systemic (e.g., intravenous) administration are required to meet strict regulatory limits on the number of particulates present within the drug vial, and these particulate limits may be exceeded if insoluble particulates are formed following drug degradation.

Liposomal formulations of camptothecin derivatives have also been reported (Emerson et al., 2000, Clinical Cancer Research 6: 2903-2912; Tardi et al., 2000, Cancer Research 60: 3389-3393). Such liposomal formulations have shown much greater antitumor activity compared with the free drug in preclinical studies and have advanced into clinical testing (Carmichael, et al., 1996, ASCO Annual Meeting, abstract no. 765; ten Bokkel Huinink et al., 1996, ASCO Annual Meeting, abstract no. 768). In such liposomal formulations almost all drug is encapsulated within the liposomes, but, nevertheless, it has surprisingly been found that drug degradation may occur with the development of insoluble precipitates overtime in the external solution of the formulation. Accordingly, there is a need in the art for the development of stable formulations of liposome-encapsulated camptothecins, for both convenience of use and increased shelf-life.

### BRIEF SUMMARY OF THE INVENTION

The present invention provides improved liposomal camptothecin compositions, formulations, and kits, as well as methods of preparing and using such compositions, formulations and kits to enhance campotothecin stability, reduce the formation and precipitation of camptothecin degradation products, and treat cancer. In various embodiments, these compositions, formulation, kits and methods include one or more features or characteristics selected from: pH of external solution is less than or equal to 4.5; empty liposomes; sphingomyelin or dihydrosphingomyelin (or a combination thereof); MnSO₄ in the internal solution; an anti-oxidant; and citrate or tartrate buffer in the external solution. As used herein, the external solution refers to solution outside of a liposome, and an internal solution refers to solution inside of a liposome. Each of these features or characteristics may be used independently, or in any combination of two or more thereof, to enhance or increase the stability of a camptothecin in a liposomal camptothecin formulation.

In one embodiment, the invention includes a liposomal formulation adapted for increased camptothecin stability, comprising a solution containing a camptothecin encapsulated in a liposome, wherein solution exterior of said liposome has a pH less than or equal to 4.5.

In another embodiment, the invention includes a liposomal formulation adapted for increased camptothecin retention and stability, comprising a solution containing a camptothecin encapsulated in a liposome, wherein solution interior of said liposome comprises MnSO₄.

In yet another embodiment, the invention includes a liposomal formulation adapted for increased camptothecin stability, comprising a solution containing a camptothecin encapsulated in a liposome, wherein said solution or liposome comprises an anti-oxidant or free radical scavenger.

In various embodiments of the present invention, the anti-oxidant or free radical scavenger is ascorbic acid. In a related embodiment, the ascorbic acid is present at a concentration in the range of 1 mM to 100 mM, and in a particular embodiment, the concentration of the ascorbic acid is approximately 10 mM. In another embodiment, the anti-oxidant is alpha-tocopherol. In a related embodiment, the alpha-tocopherol is present at a concentration in the range of 0.1 to 10 mole percent (relative to lipid), and in a particular embodiment, the alpha-tocopherol is present at a concentration in the range of 0.4 to 3 mole percent or approximately 2 mole percent.

In a further embodiment, the invention includes a liposomal formulation adapted to decrease the rate of formation of particulates, comprising a solution containing a camptothecin encapsulated in a liposome, wherein said solution further contains empty liposomes.

In an additional related embodiment, the invention includes a liposomal formulation adapted for increased camptothecin stability, comprising a solution containing a camptothecin encapsulated in a liposome, wherein said solution exterior of said liposome comprises citrate or tartrate.

In another embodiment, the invention includes a liposomal formulation adapted for increased camptothecin retention and stability, comprising a solution containing a camptothecin encapsulated in a liposome, wherein said solution exterior of said liposome has a pH less than or equal to 4.5 and wherein said solution interior of said liposome comprises MnSO₄.

In yet another embodiment, the invention includes a liposomal formulation adapted to decrease the rate of formation of particulates and increase camptothecin stability, comprising a solution containing a camptothecin encapsulated in a liposome, wherein said solution further contains empty liposomes and wherein said solution exterior of said liposome comprises citrate or tartrate.

In another embodiment, the invention includes a liposomal formulation adapted for increased camptothecin stability, comprising a solution containing a camptothecin encapsulated in a liposome wherein said solution interior of said liposome comprises MnSO₄, wherein said solution exterior of said liposome has a pH less than or equal to 4.5, and wherein said solution exterior of said liposome comprises an anti-oxidant or free radical scavenger. In a particular embodiment, the anti-oxidant or free radical scavenger is ascorbic acid.

In another embodiment, the invention includes a liposomal formulation adapted for increased camptothecin stability, comprising a solution containing a camptothecin encapsulated in a liposome, wherein said solution comprises an antioxidant or free radical scavenger and wherein the partial pressure of oxygen is lower than the atmospheric partial pressure.

In another embodiment, the present invention includes a liposomal formulation adapted for increased camptothecin stability, comprising a solution containing a camptothecin encapsulated in a liposome, wherein the external solution has a pH less than or equal to 4.5, and the solution comprises an anti-oxidant. In a related embodiment, the internal solution further comprises MnSO₄.

In another embodiment, the present invention includes a liposomal formulation adapted for increased camptothecin stability, comprising a solution containing a camptothecin encapsulated in a liposome In various embodiments, the liposomes comprise sphingomyelin (SM) and cholesterol. In further embodiments, the liposomes comprise dihydrosphingomyelin (DHSM) and cholesterol. In particular embodiments, the liposomes comprise both SM and DHSM.

In one embodiment, the present invention includes a liposomal formulation adapted for increased camptothecin stability, comprising a solution containing a camptothecin encapsulated in a liposome, wherein the exterior solution has a pH less than or equal to 4.5, and the liposome comprises DHSM. In related embodiments, the solution further comprises an anti-oxidant and/or the internal solution comprises MnSO₄.

In one particular embodiment, the present invention includes a liposomal formulation adapted for increased camptothecin stability, comprising a solution containing a camptothecin encapsulated in a liposome, wherein the exterior solution has a pH less than or equal to 4.5, the liposome comprises DHSM, the solution further comprises an anti-oxidant, and the internal solution comprises MnSO₄.

In other embodiments, the camptothecin is topotecan. In particular embodiment, the topotecan is present at a unit dosage form of about 0.01 mg/m²/dose to about 7.5 mg/m²/dose.

In a related embodiment, the invention provides a method for reducing the accumulation of camptothecin degradation products in a liposomal formulation comprising a solution containing a camptothecin encapsulated in a liposome, comprising having, adjusting to, or maintaining the pH of the solution exterior of said liposomes at or below 4.5.

In another related embodiment, the invention provides a method for reducing the accumulation of camptothecin degradation products in a liposomal formulation comprising a solution containing a camptothecin encapsulated in a liposome, comprising including MnSO₄ in the solution interior of said liposome.

An additional related embodiment of the invention provides a method for reducing the accumulation of camptothecin degradation products in a liposomal formulation comprising a solution containing a camptothecin encapsulated in a liposome, wherein said liposome comprises sphingomyelin and cholesterol, and further comprising MnSO₄ in the solution interior of said liposome.

An additional related embodiment of the invention provides a method for reducing the accumulation of camptothecin degradation products in a liposomal formulation comprising a solution containing a camptothecin encapsulated in a liposome, wherein said liposome comprises DHSM and cholesterol, comprising including MnSO₄ in the solution interior of said liposome.

The invention further provides a method for reducing the accumulation of camptothecin degradation products in a liposomal formulation comprising a solution containing a camptothecin encapsulated in a liposome, comprising including an anti-oxidant or free radical scavenger in said solution or liposome.

Additionally, the invention provides a method for reducing the accumulation of camptothecin degradation products in a liposomal formulation comprising a camptothecin encapsulated in a liposome, comprising including empty liposomes in the formulation. In one embodiment, the formulation is stored at a temperature between 2° C. and 8° C.

In another embodiment, the invention provides a method for reducing the accumulation of camptothecin degradation products in a liposomal formulation comprising a solution containing a camptothecin encapsulated in a liposome, comprising including citrate or tartrate in the solution exterior of said liposome.

The invention further provides a method for reducing the accumulation of camptothecin degradation products in a liposomal formulation comprising a solution containing a camptothecin encapsulated in a liposome, comprising including an anti-oxidant or free radical scavenger in said solution or liposome, and reducing the oxygen partial pressure in the solution to below atmospheric partial pressure.

Another related embodiment provides a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, and including an anti-oxidant in the formulation.

Another related embodiment provides a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, and including MnSO₄ in the internal solution. In a further related embodiment, the solution further comprises an anti-oxidant.

In another embodiment, the present invention includes a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, and including DHSM in the liposome.

In a related embodiment, the present invention provides a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, including DHSM in the liposome, and including an anti-oxidant in the solution.

A further embodiment provides a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, including DHSM in the liposome, and including MnSO₄ in the internal buffer.

A related embodiment includes a method for reducing the amount or accumulation of camptothecin degradation products in a liposomal formulation comprising a solution containing a camptothecin encapsulated in a liposome, comprising having the pH of the external solution of a liposomal camptothecin formulation less than or equal to 4.5, including DHSM in the liposome, including MnSO₄ in the internal buffer, and including an anti-oxidant in the solution.

In various embodiments of the methods of the invention, the camptothecin is topotecan. In particular embodiment, the topotecan is present at a unit dosage form of about 0.01 mg/m²/dose to about 7.5 mg/m²/dose.

In other embodiments of the methods of the invention, the liposome comprises sphingomyelin and cholesterol.

In other embodiments of the methods of the invention, the liposome comprises dihydrosphingomyelin and cholesterol.

According to various embodiments of the formulations, methods, and kits provided by the present invention, the solution contains not more than 3000 particles greater than 10 microns and not more than 300 particles greater than 25 microns after three months storage.

The invention further provides pharmaceutical compositions comprising a liposomal camptothecin formulation of the present invention. In one embodiment, the pharmaceutical composition is adapted for intravenous administration.

Another embodiment of the invention includes a kit comprising liposome-encapsulated camptothecin for administration to a patient in need thereof, comprising a vial comprising a solution containing a camptothecin encapsulated in a liposome, wherein said solution interior of said liposome comprises MnSO₄, and instructions for preparing the liposome-encapsulated camptothecin for administration to a patient.

A further embodiment of the invention includes a kit comprising liposome-encapsulated camptothecin for administration to a patient in need thereof, comprising a vial comprising a solution containing a camptothecin encapsulated in a liposome, wherein said solution or liposome comprises an anti-oxidant, and instructions for preparing the liposome-encapsulated camptothecin for administration to a patient.

Another embodiment of the invention provides a kit comprising liposome-encapsulated camptothecin for administration to a patient in need thereof, comprising a vial comprising a solution containing a camptothecin encapsulated in a liposome, wherein said solution further contains empty liposomes, and instructions for preparing the liposome-encapsulated camptothecin for administration to a patient.

Another related embodiment of the invention includes a kit comprising liposome-encapsulated camptothecin for administration to a patient in need thereof, comprising vial comprising a solution containing a camptothecin encapsulated in a liposome, wherein said solution exterior of said liposome comprises citrate or tartrate, and instructions for preparing the liposome-encapsulated camptothecin for administration to a patient.

In a further specific embodiment, the invention provides a kit for preparing liposome-encapsulated topotecan for administration to a patient in need thereof, comprising a first vial comprising a solution containing a liposome, wherein said liposome comprises dihydrosphingomyelin, wherein said liposome comprises encapsulated topotecan, wherein said solution interior of said liposome comprises MnSO₄, wherein said solution exterior of said liposome has a pH less than or equal to 4.0, and wherein said solution or liposome comprises ascorbic acid at concentration of 10 mM, and instructions for preparing the liposome-encapsulated topotecan for administration to a patient.

In various kit embodiments, the camptothecin is topotecan. In particular embodiments, the topotecan is present at a unit dosage form of about 0.01 mg/m²/dose to about 7.5 mg/m²/dose.

In other kit embodiments, the liposome comprises sphingomyelin and cholesterol.

In further related embodiments, the invention includes methods of treating cancer, comprising administering a liposomal formulation or pharmaceutical composition of the present invention to a patient in need thereof. In one embodiment, said patient is diagnosed with a cancer.

### BRIEF DESCRIPTION OF THE SEVERAL VIEWS OF THE DRAWING(S)

FIG. 1 provides a graphical representation of the kinetics of the appearance of topotecan crystalline particulates for 1 mg/ml liposomal topotecan samples incubated at 35° C. (A) and (B) were incubated in an external buffer of 300 mM sucrose, 10 mM citrate, pH 6, while (C) and (D) were incubated in 300 mM sucrose, 10 mM phosphate, pH 6. Data in panels (A) and (C) represent the total number of particles counted, whereas panels (B) and (D) represent the numbers of crystals observed with a length of >25 μm. Data represent an average of four 0.4 μl counts±one S.D.

FIG. 2 provides graphical depictions of the effect of temperature on topotecan crystal particulate formation in phosphate buffer. (A) Liposomal topotecan (2 mg/ml) with an external buffer of 300 mM sucrose, 10 mM phosphate, pH 6.0 was incubated in 1 ml aliquots at 5, 25 and 35° C., and samples were analyzed for crystal particulates over a five week period. (B) A semi-logarithmic plot of the results at five weeks. The dashed-line indicates the LOD using the hemocytometer technique. The data represent the average of four 0.4 μl counts±one S.D.

FIG. 3 depicts topotecan crystal particulate formation associated with various concentrations of liposomal topotecan having an external buffer of 300 mM sucrose, 10 mM phosphate, pH 6.0 and incubated at 35° C. for 3 weeks. Data represent the average of four 0.4 μl counts±one S.D.

FIG. 4 provides a semi-logarithmic plot showing the effect of the external pH on crystal numbers for liposomal topotecan (2 mg/ml) incubated at 35° C. for 5 weeks in an external buffer of 300 mM sucrose, 10 mM citrate and pH range of 3.5 to 6.0. The dashed line indicates the LOD (2000 crystals/ml) of the hemocytometer technique. Data represent the average of four 0.4 μl counts±one S.D.

FIG. 5 provides a graphical representation of the effect of different external buffers on crystal formation of liposomal topotecan (4 mg/ml) incubated at 35° C. (A) provides a comparison between 10 mM phosphate and citrate at pH 6.0 and (B) provides a comparison between 10 mM phosphate and tartrate at pH 4.0. Data represent an average of four 0.4 μl counts±one S.D.

FIG. 6 provides a graphical representation of the effect of empty liposomes on topotecan crystal particulate formation. Liposomal topotecan (0.5 mg/ml) was incubated with various amounts of empty ESM/CH (55:45 mol ratio) or POPC/CH (55:45 mol ratio) vesicles (zero to seven-fold excess lipid, w/wt) in an external buffer of 300 mM sucrose, 10 mM citrate, pH 6.0. (A) one week at 35° C., (B) two weeks at 35° C. and (C) two weeks at 25° C.

FIG. 7 provides a graph showing the effect of ascorbic acid on topotecan crystal formation, in various liposomal topotecan formulations indicated. Crystal formation was followed at 37° C. for liposomal topotecan formulation consisting of: SM/CH liposomes loaded using MgSO₄ with an external solution of 300 mM sucrose, 10 mM phosphate pH 6, ; SM/CH liposomes loaded using MgSO₄ with an external solution of 300 mM sucrose, 10 mM phosphate pH 6, 10 mM ascorbic acid, ∘; DHSM/CH liposomes loaded using MnSO₄ with an external solution of 300 mM sucrose, 10 mM phosphate pH 6, ▴; DHSM/CH liposomes loaded using MnSO₄ with an external solution of 300 mM sucrose, 10 mM phosphate pH 6, 10 mM ascorbic acid, Δ. The data are displayed as the total particulates per ml at various time points and represent an average of four 0.4 ml counts±one S.D.

FIG. 8 provides a graph showing the effect of various concentrations of alpha-tocopherol on topotecan crystal formation. Crystal formation was followed at 37° C. for liposomal topotecan formulation consisting of: DHSM/CH liposomes loaded using MnSO₄ with an external solution of 300 mM sucrose, 10 mM citrate pH 6 containing various contents of alpha-tocopherol (mole % relative to lipid); 0%, ▴; 0.2%, ∘; 0.5%, ▪; 1.0%, □; 2.0%, ∇. The data are displayed as the total particulates per ml at various time points and represent an average of four 0.4 ml counts±one S.D.

FIG. 9 provides a graph showing the decrease in ascorbic acid concentration over time for liposomal topotecan vials filled under atmospheric oxygen, ▪; and the decreased rate of ascorbic acid degradation when a nitrogen atmosphere is used ♦.

### DETAILED DESCRIPTION OF THE INVENTION

Pharmaceutical products intended to be given systemically to patients (e.g., intravenously) must meet safety and quality standards established by regulatory agencies, such as the Food and Drug Administration (FDA) in the United States, the Therapeutic Products Directorate (TPD) in Canada, and the European Medicines Agency (EMEA). Included in the quality standards set by these agencies are limits on the number of particles that can be present in the product. For example, the FDA requires that each drug vial contain not more than 3000 particles greater than 10 microns and not more than 300 particles greater than 25 microns. This limitation on particles size applies over the intended shelf-life of the product, and, hence, pharmaceutical products wherein particles are generated during storage may have a shortened commercial shelf-life. If particle formation is rapid the resulting shortened product shelf-life may make commercialization uneconomical or impractical.

It has been found that liposomal formulations of topotecan show the rapid occurrence of crystalline particulates on storage, even at 2-8° C. The occurrence of crystalline precipitates in aqueous solutions of topotecan has been described in the literature (Kearney et al., 1996). This precipitate was identified by Kearney et al. as 10-hydroxycamptothecin. Formation of topotecan dimer was also reported by Kearney et al. with this degradation product being most favored under basic conditions. In a study examining topotecan degradation in the presence of ammonium chloride, 9-aminomethyl-10-hydroxycamptothecin (9-AMT) and an N—N bis adduct (topotecan amine dimer) were identified (Patel et al., 1997, International Journal of Pharmaceutics 151, 7-13). These degradation products however were not seen in the absence of ammonium chloride. The occurrence of crystalline particulates in liposomal topotecan suspensions was unexpected as almost all drug is encapsulated within the liposomes (>98%). Further, this encapsulated topotecan is primarily in a precipitated form that confers increased drug stability. In addition, in liposomal topotecan, the crystalline particulates result from a minor degradation product, topotecan dimer, present at very low levels in the product. Surprisingly, despite the very low levels of topotecan dimer present, this hydrophobic molecule readily crystallizes, giving rise to numbers of particulates that exceed regulatory requirements. Accordingly, the shelf-life for liposomal topotecan is greatly shortened, thereby preventing clinical development and commercialization.

The present invention provides new and remarkably effective composition, formulations, methods, and kits that reduce particulate formation in suspensions of liposomal camptothecin formulations. Accordingly, the present invention provides liposomal drug formulations with increased stability and decreased degradation of the drug product, as well as reduced formation of particulate matter.

The present invention is based on the discovery of several alternative methods for reducing the formation of particulates in liposomal camptothecin formulations, each of which may be used alone or in combination with one or more other alternative methods. These inventive methods may be applied to any liposomal drug formulations, including, but not limited to the liposomes and drugs described below. In one representative embodiment, the present invention includes liposomal topotecan formulations that exhibit decreased formation of crystalline particulates in the external solution as compared to other liposomal topotecan formulations. This decreased formation of crystalline particulates confers a greatly increased product shelf-life allowing use in clinical studies and ultimately allowing commercialization.

### A. Liposomes

The methods of reducing precipitate formation in the external solution of liposomal drug formulations provided by the present invention are applicable to any type of liposome. Accordingly, the present invention includes liposomal drug formulations comprising any type of liposome known in the art, including those exemplified below. As used herein, a liposome is a structure having lipid-containing membranes enclosing an aqueous interior. Liposomes may have one or more lipid membranes. The invention includes both single-layered liposomes, which are referred to as unilamellar, and multi-layer liposomes, which are referred to as multilamellar.

1. Liposome Composition

Liposomes of the invention may include any of a wide variety of different lipids, including, e.g., amphipathic, neutral, cationic, and anionic lipids. Such lipids can be used alone or in combination, and can also include additional components, such as cholesterol, bilayer stabilizing components, e.g., polyamide oligomers (see, U.S. Pat. No. 6,320,017), peptides, proteins, detergents, and lipid-derivatives, such as PEG coupled to phosphatidylethanolamine and PEG conjugated to ceramides (see U.S. Pat. No. 5,885,613).

In numerous embodiments, amphipathic lipids are included in liposomes of the present invention. “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, Iysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Any of a number of neutral lipids can be included, referring to any of a number of lipid species which exist either in an uncharged or neutral zwitterionic form at physiological pH, including, e.g., diacylphosphatidylcholine, diacylphosphatidylethanolamine, ceramide, sphingomyelin, cephalin, cholesterol, cerebrosides, diacylglycerols, and sterols.

Cationic lipids, which carry a net positive charge at physiological pH, can readily be incorporated into liposomes for use in the present invention. Such lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 30-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL).

Anionic lipids suitable for use in the present invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In one embodiment, cloaking agents, which reduce elimination of liposomes by the host immune system, can also be included in liposomes of the present invention, such as polyamide-oligomer conjugates, e.g., ATTA-lipids, (see, U.S. patent application Ser. No. 08/996,783, filed Feb. 2, 1998) and PEG-lipid conjugates (see, U.S. Pat. Nos. 5,820,873, 5,534,499 and 5,885,613).

Also suitable for inclusion in the present invention are programmable fusion lipid formulations. Such formulations have little tendency to fuse with cell membranes and deliver their payload until a given signal event occurs. This allows the lipid formulation to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the liposome membrane over time. By the time the formulation is suitably distributed in the body, it has lost sufficient cloaking agent so as to be fusogenic. With other signal events, it is desirable to choose a signal that is associated with the disease site or target cell, such as increased temperature at a site of inflammation.

In certain embodiments, liposomes of the present invention comprises sphingomyelin (SM). As used herein, the general term sphingomyelin (SM) includes SMs having any long chain base or fatty acid chain. Naturally occurring SMs have the phosphocholine head group linked to the hydroxyl group on carbon one of a long-chain base and have a long saturated acyl chain linked to the amide group on carbon 2 of the long-chain base (reviewed in Barenholz, Y. In Physiology of Membrane Fluidity, Vol. 1. M. Shinitsky, editor. CRC Press, Boca Raton, Fla. 131-174 (1984)). In cultured cells, about 90 to 95% of the SMs contain sphingosine (1,3-dihydroxy-2-amino-4-octadecene), which contains a trans-double bond between C4 and C5, as the long-chain base, whereas most of the remainder have sphinganine (1,3-dihydroxy-2-amino-4-octadecane) as the base and lack the trans double bond between carbons 4 and 5 of the long chain base. The latter SMs are called dihydrosphingomyelins (DHSM). DHSM may contain one or more cis double bonds in the fatty acid chain. In one embodiment, DHSM contains both a fully saturated fatty acid chain and a saturated long base chain. Dihydrosphingomyelin is more specifically defined herein as any N-acylsphinganyl-1-O-phosphorylcholine derivative. Liposomes comprising SM or, specifically, DHSM, are described in further detail in U.S. Provisional Patent Application No. 60/571,712.

In a related embodiment, liposomes of the present invention comprise SM and cholesterol or DHSM and cholesterol. Liposomes comprising SM and cholesterol are referred to as sphingosomes and are further described in U.S. Pat. Nos. 5,543,152, 5,741,516, and 5,814,335. The ratio of SM to cholesterol in the liposome composition can vary. In one embodiment, it is in the range of from 75/25 (mol %/mol %) SM/cholesterol 30/70 (mol %/mol %) SM/cholesterol, 60/40 (mol %/mol %) SM/cholesterol to 40/60 (mol %/mol %) SM/cholesterol, or about 55/45 (mol %/mol %) SM/cholesterol. Generally, if other lipids are included, the inclusion of such lipids will result in a decrease in the SM/cholesterol ratio. The ratio of DHSM to cholesterol in the liposome composition can also vary. In one embodiment, it is in the range of from 75/25 (mol %/mol %) DHSM/cholesterol 30/70 (mol %/mol %) DHSM/cholesterol, 60/40 (mol %/mol %) DHSM/cholesterol to 40/60 (mol %/mol %) DHSM/cholesterol, or about 55/45 (mol %/mol %) DHSM/cholesterol. Generally, if other lipids are included, the inclusion of such lipids will result in a decrease in the DHSM/cholesterol ratio.

In certain embodiments, it is desirable to target the liposomes of this invention using targeting moieties that are specific to a cell type or tissue. Targeting of liposomes using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can comprise the entire protein or fragments thereof. A variety of different targeting agents and methods are described in the art, e.g., in Sapra, P. and Allen, T M, Prog. Lipid Res. 42(5):439-62 (2003); and Abra, R M et al., J. Liposome Res. 12:1-3, (2002).

The use of liposomes with a surface coating of hydrophilic polymer chains, such as polyethylene glycol (PEG) chains, for targeting has been proposed (Allen, et al., 1995; DeFrees, et al., 1996; Blume, et al., 1993; Klibanov, et al., 1992; Woodle, 1991; Zalipsky, 1993; Zalipsky, 1994; Zalipsky, 1995). In one approach, a ligand, such as an antibody, for targeting the liposomes is linked to the polar head group of lipids forming the liposome. In another approach, the targeting ligand is attached to the distal ends of the PEG chains forming the hydrophilic polymer coating (Klibanov et al., 1992; Kirpotin, et al., 1992).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726. Examples of targeting moieties also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

2. Method of Preparing Liposomes

A variety of methods for preparing liposomes are known in the art, including e.g., those described in Szoka, et al., Ann. Rev. Biophys. Bioeng. 9:467 (1980); U.S. Pat. Nos. 4,186,183, 4,217,344, 4,235,871, 4,261,975, 4,485,054, 4,501,728, 4,774,085, 4,837,028, 4,946,787; PCT Publication No. WO 91/17424; Deamer and Bangham, Biochim. Biophys. Acta 443:629-634 (1976); Fraley, et al., Proc. Natl. Acad. Sci. USA 76:3348-3352 (1979); Hope, et al., Biochim. Biophys. Acta 812:55-65 (1985); Mayer, et al., Biochim. Biophys. Acta 858:161-168 (1986); Williams, et al., Proc. Natl. Acad. Sci. 85:242-246 (1988); Liposomes, Marc J. Ostro, ed., Marcel Dekker, Inc., New York, 1983, Chapter 1; Hope, et al., Chem. Phys. Lip. 40:89 (1986); and Liposomes: A Practical Approach, Torchilin, V. P. et al., ed., Oxford University Press (2003), and references cited therein. Suitable methods include, but are not limited to, sonication, extrusion, high pressure/homogenization, microfluidization, detergent dialysis, calcium-induced fusion of small liposome vesicles, and ether-infusion methods, all of which are well known in the art.

Alternative methods of preparing liposomes are also available. For instance, a method involving detergent dialysis based self-assembly of lipid particles is disclosed and claimed in U.S. Pat. No. 5,976,567, which avoids the time-consuming and difficult to-scale drying and reconstitution steps. Further methods of preparing liposomes using continuous flow hydration are under development and can often provide the most effective large scale manufacturing process.

One method produces multilamellar vesicles of heterogeneous sizes (Bangham, A. and Haydon, D. A., Br Med. Bull. 24(2):124-6 (1968) and Bangham, A. D., Prog Biophys Mol Bio. 18:29-95 (1968)). In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents, such as deoxycholate.

Unilamellar vesicles can be prepared by sonication or extrusion. Sonication is generally performed with a tip sonifier, such as a Branson tip sonifier, in an ice bath. Typically, the suspension is subjected to severed sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. Unilamellar vesicles can also be made by dissolving phospholipids in ethanol and then injecting the lipids into a buffer, causing the lipids to spontaneously form unilamellar vesicles. Also, phospholipids can be solubilized into a detergent, e.g., cholates, Triton X, or n-alkylglucosides. Following the addition of the drug to the solubilized lipid-detergent micelles, the detergent is removed by any of a number of possible methods including dialysis, gel filtration, affinity chromatography, centrifugation, and ultrafiltration.

Following liposome preparation, the liposomes that have not been sized during formation may be sized to achieve a desired size range and relatively narrow distribution of liposome sizes. A size range of about 0.2-0.4 microns allows the liposome suspension to be sterilized by filtration through a conventional filter. The filter sterilization method can be carried out on a high throughput basis if the liposomes have been sized down to about 0.2-0.4 microns.

Several techniques are available for sizing liposomes to a desired size. General methods for sizing liposomes include, e.g., sonication, by bath or by probe, or homogenization, including the method described in U.S. Pat. No. 4,737,323. Sonicating a liposome suspension either by bath or probe sonication produces a progressive size reduction down to small unilamellar vesicles less than about 0.05 microns in size. Homogenization is another method that relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve gradual reduction in liposome size. Liposome size can be determined and monitored by known techniques, including, e.g., conventional laser-beam particle size discrimination or the like.

Liposomes of any size may be used according to the present invention. In certain embodiments, liposomes of the present invention have a size ranging from about 0.05 microns to about 0.45 microns, between about 0.05 and about 0.2 microns, or between 0.08 and 0.12 microns in diameter. In one embodiment, liposomes of the present invention are about 0.1 microns in diameter. In other embodiments, liposomes of the present invention are between about 0.45 microns to about 3.0 microns, about 1.0 to about 2.5 microns, about 1.5 to about 2.5 microns and about 2.0 microns.

In certain embodiments, liposomes are prepared to facilitate loading of a camptothecin into the liposomes. For example, in certain embodiments, liposomes are prepared with a pH gradient or a transmembrane potential in order to facilitate drug loading according to methods described below. Thus, in certain embodiments, the liposomes used in the present invention comprise a pH gradient across the membrane. In one embodiment, the pH is lower at the interior of the liposomes than at the exterior. Such gradients can be achieved, e.g., by formulating the liposomes in the presence of a buffer with a low pH, e.g., having a pH between about 2 and about 6, and subsequently transferring the liposomes to a higher pH solution. For example, before or after sizing of liposomes, the external pH can be raised, e.g., to about 7 or 7.5, by the addition of a suitable buffer, such as a sodium phosphate buffer. Also, in one embodiment, the liposomes used in the present invention comprise a transmembrane potential, while in another embodiment, liposomes of the invention do not comprise a transmembrane potential.

### B. Camptothecins

The present invention includes liposomal compositions comprising a camptothecin. As used herein, the term “camptothecin” includes camptothecin, as well as any and all salts, derivatives, and analogs of camptothecin. Camptothecin (CPT) compounds include various 20(S)-camptothecins, analogs of 20(S)camptothecin, and derivatives of 20(S)-camptothecin. Camptothecin, when used in the context of this invention, includes the plant alkaloid 20(S)-camptothecin, both substituted and unsubstituted camptothecins, and analogs thereof. Examples of camptothecin derivatives include, but are not limited to, 9-nitro-20(S)-camptothecin, 9-amino-20(S)-camptothecin, 9-methyl-camptothecin, 9-chlorocamptothecin, 9-fluoro-camptothecin, 7-ethyl camptothecin, 10-methylcamptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, 7-(4-methylpiperazinomethylene)-10,11-methylenedioxy-20(S)-camptothecin, 7-(4-methylpiperazinomethylene)-10,11-ethylenedioxy-20(S)-camptothecin, and 7-(2-N-isopropylamino)ethyl)-(20S)-camptothecin (also termed CKD-602). Prodrugs of camptothecin include, but are not limited to, esterified camptothecin derivatives as described in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate. Particular examples of 20(S)-camptothecins include 9-nitrocamptothecin, 9-aminocamptothecin, 10,11-methylendioxy-20(S)camptothecin, topotecan, irinotecan, 7-ethyl-10-hydroxy camptothecin, or another substituted camptothecin that is substituted at least one of the 7, 9, 10, 11, or 12 positions. These camptothecins may optionally be substituted, e.g., at the 7, 9, 10, 11, and/or 12 positions. Such substitutions may serve to provide differential activities over the unsubstituted camptothecin compound. Examples of substituted camptothecins include 9-nitrocamptothecin, 9-aminocamptothecin, 10,11-methylendioxy-20(S)-camptothecin, topotecan, irinotecan, exatecan, 7-ethyl-10-hydroxy camptothecin, or another substituted camptothecin that is substituted at least one of the 7, 9, 10, 11, or 12 positions.

Topotecan is a semisynthetic structure analog of camptothecin. It is water-soluble and contains an intact lactone ring, which may open in a reversible, pH-dependent reaction, forming a carboxylate derivative. Below pH 4, no open form is present, while above pH 9, more than 95% is hydrolyzed. Only the lactone form is pharmacologically active and inhibits cancer cell growth by inhibiting topoisomerase 1, an enzyme crucial for DNA replication. It has recently been discovered that the anti-tumor activity of topotecan hydrochloride (Hycamtin™, SmithKline Beecham) encapsulated in SM/cholesterol liposomes, such as SM/cholesterol (55:45) liposomes, by a gradient loading method provides surprising anticancer efficacy at lower doses, and with lower collateral toxicity, than free topotecan (described in U.S. patent application Ser. No. 09/896,811). In one embodiment, the camptothecin is topotecan, or a salt or derivative thereof.

Camptothecin derivatives may be therapeutically active themselves or they may be prodrugs, which become active upon further modification. Thus, in one embodiment, a camptothecin derivative retains some or all of the therapeutic activity as compared to the unmodified agent, while in another embodiment, a camptothecin derivative lacks therapeutic activity in the absence of further modification.

Camptothecins may give rise to degradation products that form precipitates or particulates, the rate of formation of which is reduced by the compositions and methods disclosed herein. The present invention provides compositions and methods for reducing the formation and/or accumulation of precipitates in the external solution of liposomal drug formulations. Accordingly, in certain embodiments, the present invention is particularly useful for degradation products or contaminants of camptothecins that precipitate in the external solution when present in liposomal formulations. Such precipitation may be caused by any of a variety of factors, including, e.g., the pH of the external solution and oxidative processes, and may be associated with leakage of the camptothecin from liposomes during storage. Accordingly, the present invention includes, in certain embodiment, liposomal compositions comprising a camptothecin that precipitates in the external solution, a camptothecin that undergoes oxidation, a camptothecin that undergoes pH-dependent degradation or precipitation, or a camptothecin that leaks from liposomes. For example, in one embodiment, the invention contemplates camptothecins that are not stable in the external solution. Such characteristics of drugs are generally known in the art and are described in the literature, including, e.g., King, R. E., Remington's Pharmaceutical Sciences, 17^{th} Ed., Mack Publishing Co., Philadelphia, Pa., 1985.

Liposomal topotecan compositions that may be modified or prepared as a formulation having reduced particulate formation according to the present invention described herein include, e.g., those described in U.S. patent application Ser. No. 09/896,811. In particular embodiments, the present invention provides a liposomal topotecan formulation comprising a unit dosage form of about 0.01 mg/M²/dose to about 7.5 mg/M²/dose and having a drug:lipid ratio (by weight) of about 0.05 to about 0.2. In certain aspects, the drug:lipid ratio (by weight) is about 0.05 to about 0.15. In another aspect, the liposomaltopotecan unit dosage form is about 1 mg/M²/dose to about 4 mg/M²/dose of topotecan.

Native, unsubstituted, camptothecin can be obtained by purification of the natural extract, or may be obtained from the Stehlin Foundation for Cancer Research (Houston, Tex.). Substituted camptothecins can be obtained using methods known in the literature, or can be obtained from commercial suppliers. For example, 9-nitrocamptothecin may be obtained from SuperGen, Inc. (San Ramon, Calif.), and 9-aminocamptothecin may be obtained from Idec Pharmaceuticals (San Diego, Calif.). Camptothecin and various analogs may also be obtained from standard fine chemical supply houses, such as Sigma Chemicals. Topotecan (Hycamtin) is commercially available from Smithkline Beecham (Middlesex, United Kingdom) or can be synthesized from camptothecin as described by Kingsbury et al., 1991, J. Med. Chem. 34: 98-107.

### C. Methods of Loading Liposomes

Liposomal formulations of the invention are generally prepared by loading an camptothecin into liposomes. Loading may be accomplished by any means available in the art, including those described in further detail below. Furthermore, the invention contemplates the use of either passive or active loading methods.

Passive loading generally requires addition of the drug to the buffer at the time the liposomes are formed or reconstituted. This allows the drug to be trapped within the liposome interior, where it will remain if it is not lipid soluble and if the vesicle remains intact (such methods are described, e.g., in PCT Publication No. WO 95/08986).

In one particular passive loading technique, the drug and liposome components are dissolved in an organic solvent in which all species are miscible and concentrated to a dry film. A buffer is then added to the dried film and liposomes are formed having the drug incorporated into the vesicle walls. Alternatively, the drug can be placed into a buffer and added to a dried film of only lipid components. In this manner, the drug will become encapsulated in the aqueous interior of the liposome. The buffer which is used in the formation of the liposomes can be any biologically compatible buffer solution of, for example, isotonic saline, phosphate buffered saline, or other low ionic strength buffers. The resulting liposomes encompassing the camptothecin can then be sized as described above.

Liposomal compositions of the invention may also be prepared using active loading methods. Numerous methods of active loading are known to those of skill in the art. Such methods typically involve the establishment of some form of gradient that draws lipophilic compounds into the interior of liposomes where they can reside for as long as the gradient is maintained. Very high quantities of the desired camptothecin can be obtained in the interior. At times, the camptothecin may precipitate out in the interior and generate a continuing uptake gradient. A wide variety of camptothecins can be loaded into liposomes with encapsulation efficiencies approaching 100% by using active loading methods involving a transmembrane pH or ion gradient (see, Mayer, et al., Biochim. Biophys. Acta 1025:143-151 (1990) and Madden, et al., Chem. Phys. Lipids 53:37-46 (1990)).

Transmembrane potential loading has been described in detail in U.S. Pat. Nos. 4,885,172; 5,059,421; 5,171,578; and 5,837,282 (which teaches ionophore loading). Briefly, the transmembrane potential loading method can be used with essentially any camptothecin, including, e.g., conventional drugs, that can exist in a charged state when dissolved in an appropriate aqueous medium. In certain embodiments, the camptothecin will be relatively lipophilic and will partition into the liposome membranes. A transmembrane potential is created across the bilayers of the liposomes or protein-liposome complexes and the camptothecin is loaded into the liposome by means of the transmembrane potential. The transmembrane potential is generated by creating a concentration gradient for one or more charged species (e.g., Na⁺, K⁺, and/or H⁺) across the membranes. This concentration gradient is generated by producing liposomes having different internal and external media and has an associated proton gradient. Camptothecin accumulation can then occur in a manner predicted by the Henderson-Hasselbach equation.

One particular method of loading camptothecins, including, e.g., topotecan, to produce a liposomal composition of the present invention is ionophore-mediated loading, as disclosed and claimed in U.S. Pat. No. 5,837,282. One example of an ionophore used in this procedure is A23187. With hydrogen ion transport into the vesicle, there is concomitant metal ion transport out of the vesicle in a 2:1 ratio (i.e., no net charge transfer). As ionophore-mediated loading is an electroneutral process, there is no transmembrane potential generated.

Accordingly, the invention provides methods of loading liposomes via ionophore-mediated loading. Similarly, the invention provides methods of preparing or manufacturing a liposomal composition of the invention comprising loading a liposome comprising DHSM with a camptothecin according to the method of loading liposomes described here, including ionophore-mediated loading.

In additional embodiments, the loading is performed at a temperature of at least 60° C., at least 65° C., or at least 70° C. In particular embodiments, loading is performed at a temperature in the range of 60° to 70°, and in certain embodiments, loading is performed at either 60° C. or 70° C. Loading may be performed in the presence of any concentration of camptothecin (e.g., drug), or at any desired drug to lipid ratio, including any of the drug to lipid ratios described herein. In certain embodiment, loading is performed at a drug to lipid ratio within the range of 0.005 drug:lipid (by weight) to about 1.0 drug:lipid (by weight). In particular embodiments, loading is performed at a drug to lipid ratio within the range of 0.4 drug:lipid (by weight) to 1.0 drug:lipid (by weight). In other particular embodiments, loading is performed at a drug to lipid ratio of either 0.4 drug:lipid (by weight) or 1.0 drug:lipid (by weight).

The final drug:lipid ratio of the final liposomal formulations of the present invention encompasses a wide range of suitable ratios, which can be formulated by techniques available in the art, including, e.g.: 1) using homogenous liposomes each containing the same drug:lipid ratio; or 2) by mixing empty liposomes with liposomes having a high drug:lipid ratio to provide a suitable average drug:lipid ratio. For different applications, different drug:lipid ratios may be desired. Drug:lipid ratios can be measured on a weight to weight basis, a mole to mole basis or any other designated basis. In certain embodiments, drug:lipid ratios range from about 0.005 drug:lipid (by weight) to about 0.2 drug:lipid (by weight), from about 0.01 to about 0.2 drug:lipid (by weight), from about 0.01 to about 0.05 drug:lipid (by weight), from about 0.01 drug:lipid (by weight) to about 0.02 drug:lipid (by weight). In other embodiments, drug:lipid ratios range from about 0.005 to about 0.5 (by weight), from about 0.01 to about 0.4 (by weight), from about 0.05 to about 0.4 (by weight), from about 0.05 to about 0.3 (by weight), and from about 0.1 to about 0.4 (by weight). In further embodiments, drug:lipid ratios range from about 0.01 to about 1.0, from about 0.05 to about 1.0, from about 0.1 to about 1.0, and from about 0.5 to about 1.0 (by weight). In other embodiments, the drug:lipid ratio is at least 0.01, at least 0.05, at least 0.1, at least 0.2, at least 0.3, at least 0.4, at least 0.5, at least 0.6, at least 0.7, at least 0.8, at least 0.9 or at least 1.0 (by weight).

The present invention also provides methods of preparing liposomal compositions and methods of making or manufacturing liposomal compositions of the present invention. In general, such methods comprise loading a liposome of the present invention with an camptothecin. Loading may be accomplished by any means available in the art, including those described herein, and, particularly, ionophore-mediated loading methods described here. Such methods may further comprise formulating the resulting composition to produce a pharmaceutical composition suitable for administration to a subject.

In one embodiment, the liposomes used in the present invention comprise a transmembrane potential, while in another embodiment, liposomes of the invention do not comprise a transmembrane potential.

### D. Compositions and Methods for Reducing External Solution Particulate Formation

The present invention provides compositions, formulations, and methods for reducing particulate or crystal formation, or enhancing camptothecin stability, in the external solution of liposomal camptothecin formulations, including, e.g., liposomal topotecan formulations. Features of these methods and formulations may be used alone or in combination to reduce the amount of particulate formation, the rate of particulate formation, and/or the size of particulates formed. Accordingly, in related embodiments, the invention includes liposomal compositions comprising a camptothecin and one or more of the features provided below.

Topotecan HCl, itself, is considered relatively stable in solution, although degradation products form over time (Kramer and Thiesen, Journal of Oncology Pharmacy Practice 5:75-82 (1999)). However, according to the present invention, it was surprisingly discovered that liposomal topotecan formulations accumulate crystalline precipitates in the external solution over time, including when stored at 2-8° C. The amount of crystal particulates found in the external solution of liposomal topotecan formulations containing less than 1% topotecan degradants can be enough to fail the USP particulate test within less than one year. It was further discovered that these crystalline precipitates comprise the topotecan degradation product topotecan dimer (also referred to as SKF-107030), which is different from the carboxylate derivative of toptoecan described above. Although not wishing to be bound by any particular theory, it is now believed that the degradation process that produces topotecan dimer is pH-dependent and involves an oxidation or free-radical mechanism. Accordingly, in one embodiment, the present invention includes a liposomal composition comprising topotecan and an antioxidant or free radical scavenger.

In certain embodiments, compositions and methods of the invention display an at least two-fold, five-fold, ten-fold, twenty-fold, thirty-fold, forty-fold, fifty-fold, one hundred-fold, two-hundred-fold, five-hundred-fold or one thousand-fold reduction in the number of crystals detected in the external solution by any available method, including the methods described herein, at any time point following loading of the liposomes with the camptothecin and under any temperature as compared to liposomal compositions that do not include one or more of the features described herein as enhancing stability of camptothecins in liposomal formulations.

1. Low pH External Solutions

As described below in Example 1 it was found that crystalline particulates developed in liposomal topotecan formulations on storage. It was surprisingly found that the rate of particulate formation could be remarkably reduced when the external pH was about pH 4.5 or below. Accordingly, the present invention includes liposomal formulations comprising an camptothecin and having an external solution of low pH. As described in Examples 1 and 2, this aspect of the present invention is based on the remarkable and unexpected discovery that reducing the pH of the external solution results in a surprisingly large decrease in particulate formation.

Without wishing to be bound to any particular theory, it is possible that camptothecins undergo less degradation and/or particulate formation at pHs wherein they are more soluble. Accordingly, the invention further includes liposomal compositions comprising an camptothecin wherein the pH of the external solution is a pH in which the camptothecin is soluble or wherein the camptothecin undergoes decreased degradation, as compared to certain other pHs. In one embodiment, the pH of the external solution is within 1, 2, or 3 pH units of the pH at which an camptothecin is most soluble or undergoes the least degradation. Certain methods of loading liposomes with camptothecins, including pH-gradient-mediated loading, described herein, involve generating a pH gradient across the liposomal membrane, e.g., such that the pH is lower on the inside and higher on the outside of the liposomes. This pH gradient drives the camptothecin present in the exterior solution into the interior of the liposomes. Typically, the pH of the exterior solution following loading is neutral or basic. In light of the surprising finding of the present invention that less precipitates are formed in the external solution when the pH is lower, the present invention provides a method of preparing liposomal compositions comprising an camptothecin, which involves loading liposomes with an camptothecin according to standard pH gradient-mediated, transmembrane potential-mediated or ionophore-mediated loading techniques, followed by reducing the pH of the external solution. The pH of the external buffer may be reduced by any of a variety of routine methods, including, e.g., adding an acidic buffer to the external solution or replacing the external solution with a solution having a lower pH.

In particular embodiments, the invention includes a liposomal formulation comprising a camptothecin and having an external solution pH of less than 6.0 or, preferably, less than or equal to 4.5. In one embodiment, the camptothecin is topotecan. In particular embodiments, the pH is less than or equal to 4.5, 4.2, 4.0, 3.8, 3.5, 3.2, or 3. In other embodiments, the pH is in the range between and including pH 3 and 4 or between and including pH 3 and 4.5. In another embodiment, the liposome comprises sphingomyelin and cholesterol. In a further embodiment, the liposome comprises DHSM and cholesterol.

2. Citrate and Tartrate Buffers

The present invention also provides compositions and methods related to the surprising finding that external solution buffer composition remarkably effects precipitate formation, as described in Example 2. Accordingly, the present invention includes a method for reducing particulate formation in the external solution of liposomal compositions comprising a camptothecin, e.g., topotecan, comprising using citrate or tartrate buffers in the external solution.

In a related embodiment, the present invention includes a liposomal formulation comprising liposomes having encapsulated therein an camptothecin, wherein the external solution of said liposomes is a citrate or tartrate buffer. The citrate or tartrate buffer may be present at any pH or concentrations. In certain embodiments, therefore, the pH of the citrate or tartrate-buffered external solution is acidic or neutral. In particular embodiments, the pH of the external solution is pH 6 or less or about pH 6 to about pH 7.5. In particular embodiments, the pH is about 3, about 3.5, about 4, about 4.5, about 5, about 5.5, or about 6. In one embodiment, the pH is at or between 3 and 6. In one embodiment, the liposomal formulation contains citrate buffer at a pH of approximately pH 4.0 or tartrate buffer at approximately pH 4.0. The concentration of the citrate or tartrate buffer may vary, but in certain embodiments, the concentration is less than or equal to 100 mM or greater than or equal to 1 mM. In particular embodiments, the concentration is 1-100 mM, 1-10 mM, 10-20 mM, 10-50 mM or 10-100 mM. In one particular embodiment, the concentration is about 10 mM.

Liposomal formulations having an external citrate or tartrate buffer can be readily prepared as described herein. For example, following loading of liposomes with an camptothecin, the external buffer used for loading may be replaced with a citrate or tartrate buffer of the preferred pH by routine methods.

3. Empty Liposomes

A further related aspect of the invention provides a method of reducing particulate formation by including empty liposomes in liposomal formulations comprising liposome-encapsulated camptothecin. It is a surprising finding of the present invention that including empty vesicles in liposomal formulations results in decreased formation of precipitates in the external solution, as described in Example 3. Without wishing to be bound by theory, it is believed that empty vesicles serve as sinks that collect hydrophobic degradation products thereby preventing the precipitation or crystallization of these degradation products in the external solution.

Accordingly, the present invention includes a method of reducing particulate formation in the external medium of liposomal formulations comprising a camptothecin, which includes adding empty vesicles to the liposomal formulation. In addition, the present invention includes liposomal compositions comprising liposomes containing a camptothecin and empty liposomes. Liposomal compositions comprising both loaded and empty vesicles are described in further detail, e.g., in U.S. patent application Ser. No. 10/788,649.

According to the present invention, the empty liposomes may contain the same and/or different lipid constituents than the loaded vesicles. In addition, the empty liposomes may be the same or of similar size as the loaded liposomes. Empty liposomes may be present in liposomal formulations of the invention at a wide range of different ratios as compared to loaded liposomes. For example, the ratio of empty liposomes to loaded liposomes, in certain embodiments, is less than or equal to 1:1, less than or equal to 3:1, or less than or equal to 10:1 (lipid wt/wt). In other embodiments, the ratio of empty liposomes to loaded liposomes is greater than or equal to 1:1, greater than or equal to 3:1, or greater than or equal to 10:1 (lipid wt/wt). In particular embodiments, the ratios of empty liposomes to loaded liposomes are approximately 1:1, 3:1 or 7:1 (lipid wt/wt).

4. Antioxidants and Free Radical Scavengers

Another surprising finding of the present invention is that the presence of antioxidants or free radical scavengers in liposomal formulations dramatically reduces particulate formation in the external solution, as demonstrated in Example 4. Accordingly, the present invention provides a method of reducing particulate formation in the external solution of liposomal formulations comprising adding an antioxidant to the liposomal formulation. In addition, the present invention includes liposomal formulations comprising an camptothecin and an antioxidant. The antioxidant may be present in the interior of the liposomes, incorporated into the lipid layer of the liposome, or present in the exterior solution of the liposomal formulation. Generally, hydrophobic antioxidants are present in the lipid bilayer, and hydrophilic antioxidants are present in the interior space or external solution.

A variety of antioxidants may be used according to the present invention, including, but not limited to, ascorbic acid (vitamin C), alpha-tocopherol (α-tocopherol), beta carotene (vitamin A) and other carotenoids (e.g., lutein), and selenium. Antioxidants may be included within liposomal formulations at a variety of different concentrations. For example, in various embodiment, alpha-tocopherol is included in the membrane of liposomes at a concentration less than or equal to 1 mole percent (relative to lipid), less than or equal to 2 mole percent, or less than or equal to 5 mole percent.

In addition to including antioxidants within liposomal formulations, the present invention further provides methods of reducing particulate formation or reducing oxidation of a camptothecin by other means, including, but not limited to, reducing the partial pressure of oxygen in the solution by purging with nitrogen and/or sealing the vialed liposomal formulation under a nitrogen atmosphere with an oxygen content less than that of atmospheric air. In particular embodiments, the oxygen content is less than or equal to 15%, 10%, 8%, 5%, 4%, or 3%.

5. MnSO₄

As described below in Example 5, it was a surprising finding of the present invention that the use of certain salts in the interior of liposomes comprising a camptothecin resulted in decreased precipitate formation in the external solution. The present invention, therefore, includes a method of reducing particulate formation in the exterior solution by including in the interior of the liposomes a salt or divalent cation that reduces particulate formation, such as, e.g., MnSO₄ or Mn²⁺.

In addition, the present invention provides liposomal compositions comprising a camptothecin and MnSO₄ or Mn²⁺ in the interior of the liposomes. In a related embodiment, the salt or divalent cation in the interior of liposomes comprising a camptothecin is MnSO₄ or Mn²⁺. In one embodiment, the camptothecin is topotecan, and the present invention includes a liposomal composition comprising topotecan and MnSO₄ or Mn²⁺ in the interior of the liposomes. In a preferred embodiment, the liposomes comprise sphingomyelin and cholesterol. In a further preferred embodiment, the liposomes comprise DHSM and cholesterol.

Liposomal compositions comprising MnSO₄ or Mn²⁺ can be prepared essentially as known in the art, and as described in Example 1, by substituting MnSO₄ for other salts, such as MgSO₄.

6. Lipid Components

A surprising discovery of the present invention, described in Example 5, is that the particular lipid components of liposomal formulations comprising a camptothecin effect the amount of particulate formed in the external solution. Accordingly, the present invention includes liposomal compositions including particular lipid components. In one embodiment, the liposomes of such liposomal compositions comprise dihydrosphingomyelin (DHSM).

In one embodiment, the present invention includes liposomal formulations comprising a camptothecin encapsulated in liposomes comprising DHSM and cholesterol. In a particular embodiment, the camptothecin is topotecan. Such liposomal formulations may be prepared as described, e.g., in U.S. patent application Ser. No. 09/896,811.

7. Combinations

In addition to the compositions and methods of reducing particulate formation described above, the present invention further includes liposomal formulations and methods that combine two or more features described above as reducing particular formation and enhancing camptothecin stability, preferably to achieve an even greater reduction in the amount of particulate formed in the external solution. Each of the various formulations described below may further comprise a camptothecin, such as, e.g., topotecan. In addition, in particular embodiment, each of the formulations described below may be held or stored under reduced oxygen conditions, including any of those described above.

Accordingly, in one embodiment, the invention includes compositions and methods of reducing particulate formation in the external solution comprising formulating liposomal compositions having MnSO₄ as the internal salt, in addition to using a citrate or tartrate external buffer, using a low pH external buffer (e.g., pH less than or equal to 4.5), including empty liposomes in the final formulation, using liposomes comprising SM or DHSM, and/or including an antioxidant in the liposomal formulation.

In another embodiment, the present invention includes compositions and methods of reducing particulate formation in the external solution comprising formulating liposomal compositions having a citrate or tartrate buffer in the external solution, in addition to using MnSO₄ as the internal salt, using a low pH external buffer (e.g., pH less than or equal to 4.5), including empty liposomes in the final formulation, using liposomes comprising SM or DHSM, and/or including an antioxidant in the liposomal formulation.

In another embodiment, the present invention includes compositions and methods of reducing particulate formation in the external solution comprising formulating liposomal compositions having a low pH external buffer (e.g., pH less than or equal to 4.5), in addition to using MnSO₄ as the internal salt, using a citrate or tartrate external buffer, including empty liposomes in the final formulation, using liposomes comprising SM or DHSM, and/or including an antioxidant in the liposomal formulation.

In another embodiment, the present invention includes compositions and methods of reducing particulate formation in the external solution comprising formulating liposomal compositions having empty liposomes in the final formulation, in addition to using MnSO₄ as the internal salt, using a citrate or tartrate external buffer, using a low pH external buffer (e.g., pH less than or equal to 4.5), using liposomes comprising SM or DHSM, and/or including an antioxidant in the liposomal formulation.

In another embodiment, the present invention includes compositions and methods of reducing particulate formation in the external solution comprising formulating liposomal compositions comprising SM or DHSM, in addition to using MnSO₄ as the internal salt, using a citrate or tartrate external buffer, using a low pH external buffer (e.g., pH less than or equal to 4.5), having empty liposomes in the final formulation, and/or including an antioxidant in the liposomal formulation.

In another embodiment, the present invention includes compositions and methods of reducing particulate formation in the external solution comprising formulating liposomal compositions including an antioxidant in the liposomal formulation, in addition to using MnSO₄ as the internal salt, using a citrate or tartrate external buffer, using a low pH external buffer (e.g., pH less than or equal to 4.5), using liposomes comprising SM or DHSM, and/or having empty liposomes in the final formulation.

8. Kits

In addition to providing superior liposomal formulations comprising an camptothecin and having decreased particulate formation in the external solution, the present invention allows liposomes loaded with a camptothecin to be stored for an increased length of time before administration to a patient. Accordingly, in certain embodiments, the invention provides kits comprising a liposomal formulation of a camptothecin for administration to a patient. Such kits may comprises liposomes preloaded with one or more camptothecins, e.g., topotecan, or, alternatively, such kits may include liposomes and camptothecin separately.

The compositions and methods provided herein, which reduce the amount of particulate formation in the external solution, may be incorporated into kits to provide liposomal formulations of camptothecins, wherein said formulations have an increased stability and shelf-life as compared to liposomal formulations that do not include one or more of the features described herein to external precipitate formation.

In particular embodiments, liposomal camptothecin compositions, solutions, formulations, and kits of the present invention contain not more than 3000 particles greater than 10 microns and not more than 300 particles greater than 25 microns after three months storage at room temperature or at 4° C. In related embodiments, they contain not more than 2500, 2000, 1500, 1000, 500, 300, 200, 100, or 50 particles greater than 10 microns and not more than 200, 100, or 50 particles greater than 25 microns after three months storage at room temperature or at 4° C.

In one embodiment, a kit of the present invention comprises a vial comprising a solution of liposome-encapsulated camptothecin, wherein the oxygen content of the solution is reduced as compared to atmospheric air oxygen levels. In particular embodiments, the oxygen content is less than or equal to 15%, 10%, 8%, 5%, 4%, or 3%. In one embodiment, the partial pressure of oxygen in the solution is reduced by purging with nitrogen and/or sealing the vialed liposomal formulation under a nitrogen atmosphere.

In certain embodiments, kits of the present invention comprise a formulation that requires additional preparation and/or mixing before administration. The kit will typically comprise a container that is compartmentalized for holding the various elements of the kit. For example, different compartments of a kit may each hold a vial comprising a component of the kit. In certain embodiments, the kits contain the liposomal formulations of the present invention or the components thereof, in hydrated or dehydrated form, with instructions for their rehydration, preparation, and/or administration. In one embodiment, a first vial comprises a solution comprising a liposome-encapsulated camptothecin, and a second vial comprising empty liposomes.

In one embodiment, a kit comprises one or more vials comprising a liposome-encapsulated camptothecin, as well as instructions for further preparation, or use thereof. In particular embodiments, a vial comprises a unit dosage of a camptothecin. In certain embodiments, the liposomal camptothecin unit dosage comprises a camptothecin dosage of from about 0.015 mg/m²/dose to about 1 mg/m²/dose. In one embodiment, the unit dosage form comprises a camptothecin dosage of from about 0.15 mg/m²/dose to about 0.5 mg/m²/dose. In one embodiment, the vial comprises a topotecan unit dosage form of about 0.01 mg/m²/dose to about 7.5 mg/m²/dose. In another embodiment, the liposomal topotecan unit dosage form is about 1 mg/m²/dose to about 4 mg/m²/dose of topotecan.

In particular embodiments, a kit comprises a first vial comprising liposomes and a second vial comprising a camptothecin to be loaded into the liposomes. In particular embodiments, such kits further comprise one or more vials comprising a reagent or buffer related to a particular solution to precipitate formation described herein. For example, a kit may further comprise a vial containing a solution or buffer at low pH (e.g., pH 6.0 or less or pH 4.5 or less), a citrate or tartate buffer, empty liposomes, MnSO₄ or Mn⁺, and/or an antioxidant or free radical scavenger. Alternatively, a reagent or buffer related to a particular solution to precipitate formation, as described herein, is incorporated into the first vial comprising the liposomes or the second vial comprising the camptothecin.

In particular embodiments, a kit comprises at least one vial comprising a liposome loaded with a camptothecin and incorporating one or more of the features to reduce precipitate formation described above. Of course, it is understood that any of these kits may comprise additional vials, e.g., a vial comprising a buffer, such as those described in U.S. patent application Ser. No. 10/782,738. In addition, kits may comprise instructions for the preparation and/or use of the liposomal formulations of the present invention.

In one embodiment, a kit of the present invention comprises a liposomal formulation comprising a liposome containing a camptothecin and containing MnSO₄ or Mn²⁺ in the interior of the liposomes. In a related embodiment, the camptothecin is provided separately from the liposomes.

In another embodiment, a kit of the present invention comprises a liposomal formulation comprising a liposome containing a camptothecin, wherein the external solution comprises a citrate or tartrate buffer. In a related embodiment, the camptothecin is provided separately from the liposomes.

In a related embodiment, a kit of the present invention comprises a liposomal formulation comprising a liposome containing a camptothecin, wherein the external solution has a pH equal of less than 6.0 or less than or equal to 4.5. In a related embodiment, the camptothecin is provided separately from the liposomes.

In a further embodiment, a kit of the present invention comprises a liposomal formulation comprising a liposome containing a camptothecin, wherein said liposome comprises SM or DHSM. In a related embodiment, the camptothecin is provided separately from the liposomes.

In another embodiment, a kit of the present invention comprises a liposomal formulation comprising a liposome containing a camptothecin and an antioxidant or free radical scavenger. In a related embodiment, the camptothecin is provided separately from the liposomes.

In another embodiment, a kit of the present invention comprises a liposomal formulation comprising liposomes containing a camptothecin and empty liposomes.

It is understood that kits of the present invention may incorporate any of the liposomal camptothecin formulations or solutions provided herein, and various combinations thereof. Thus, in another embodiment, e.g., a kit of the present invention comprises a liposome containing an antioxidant and having MnSO₄ or Mn²⁺ in the interior of the liposome, a camptothecin, and a buffer or solution having a pH of less than 6.0 or less than or equal to 4.5. In another related embodiment, a kit of the present invention comprises a liposome containing an antioxidant and a camptothecin, and an additional compartment containing a solution having a pH less than or equal to 6.0 or less than or equal to 4.5. The camptothecin may be present within the liposomes or provided separately.

In one embodiment, a kit comprises a liposome comprising DHSM, which further contains an antioxidant and has MnSO₄ or Mn²⁺ in the interior of the liposome, a camptothecin, and a buffer or solution having a pH of less than 6.0 or less than or equal to 4.5. In a particular embodiment, the camptothecin is present within the liposome.

In a specific embodiment directed to topotecan, a kit comprises liposomes comprising DHSM and having MnSO₄ or Mn²⁺ in the interior of the liposome, wherein said liposome further comprises ascorbic acid at a concentration of 10 mM and contains topotecan, wherein the exterior solution of the liposome has a pH of less than or equal to 6.0, less than or equal to 4.5, or approximately 4.0.

In another related embodiment, a kit comprises a first vial containing a liposome comprising DHSM and having MnSO₄ or Mn²⁺ in the interior of the liposome, wherein said liposome further comprises ascorbic acid at a concentration of approximately 10 mM, and further comprises encapsulated toptoecan. The kit may optionally comprise a second vial containing a buffered solution having a pH of less than 6.0 or less than or equal to 4.5, including but not limited to, approximately 4.0.

In another embodiment, a kit comprises a first vial containing a liposome comprising a camptothecin, e.g., topotecan, and a second vial comprising an antioxidant or free radical scavenger.

Kits of the present invention that provide the camptothecin separately from the liposomes may further include an ionophore suitable for ionophore-mediated loading of the camptothecin into the liposomes.

### E. Liposomal Delivery of Camptothecins

The liposomal compositions described above may be used for a variety of purposes, including the delivery of a camptothecin to a subject or patient in need thereof. Subjects include both humans and non-human animals. In certain embodiments, subjects are mammals. In other embodiments, subjects are one or more particular species or breed, including, e.g., humans, mice, rats, dogs, cats, cows, pigs, sheep, or birds.

Thus, the present invention also provides methods of treatment for a variety of diseases and disorders, including but not limited to tumors, comprising administering a liposomal camptothecin formulation of the present invention to a patient in need thereof.

1. Methods of Treatment

The liposomal compositions of the present invention may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, muscoloskeletal diseases, neurological diseases, neuromuscular diseases, metabolic diseases, sexually transmitted diseases, skin and connective tissue diseases, urological diseases, and infections.

In one embodiment, the liposomal compositions and methods described herein can be used to treat any type of tumor or cancer. In particular, these methods can be applied to ovarian cancer, small cell lung cancer, non-small cell lung cancer, colorectal cancer and cancers of the blood and lymphatic systems, including lymphomas, leukemia, and myelomas. The compositions and methods described herein may also be applied to any form of leukemia, including adult and childhood forms of the disease. For example, any acute, chronic, myelogenous, and lymphocytic form of the disease can be treated using the methods of the present invention. In preferred embodiments, the methods are used to treat Acute Lymphocytic Leukemia (ALL). More information about the various types of leukemia can be found, inter alia, from the Leukemia Society of America (see, e.g., www.leukemia.org).

Additional types of tumors can also be treated using the methods described herein, such as neuroblastomas, myelomas, prostate cancers, brain tumors, breast cancer, and others.

The liposomal compositions of the invention may be administered as first line treatments or as secondary treatments. In addition, they may be administered as a primary chemotherapeutic treatment or as adjuvant or neoadjuvant chemotherapy. For example, treatments of relapsed, indolent, transformed, and aggressive forms of non-Hodgkin's Lymphoma may be administered following at least one course of a primary anti-cancer treatment, such as chemotherapy and/or radiation therapy, followed by at least one partial or complete response to the at least one treatment.

2. Administration of Liposomal Compositions

Liposomal compositions of the invention are administered in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered intravenously or intraarterially either by bolus injection or by infusion. For example, in one embodiment, a patient is given an intravenous infusion of the liposome-encapsulated camptothecin through a running intravenous line over, e.g., 5-10 minutes, 15-20 minutes, 30 minutes, 60 minutes, 90 minutes, or longer. In one embodiment, a 60 minute infusion is used. In other embodiments, an infusion ranging from 6-10 or 15-20 minutes is used. Such infusions can be given periodically, e.g., once every 1, 3, 5, 7, 10, 14, 21, or 28 days or longer, preferably once every 7-21 days, and preferably once every 7 or 14 days. As used herein, each administration of a liposomal composition of the invention is considered one “course” of treatment.

Liposomal compositions of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. In one embodiment, the present invention provides pharmaceutical compositions formulated for any particular route of delivery, including, e.g., intravenous administration. Methods of formulating pharmaceutical compositions for different routes of administration are known in the art.

The concentration of liposomes in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration can be increased to lower the fluid load associated with treatment. Alternatively, liposomes composed of irritating lipids can be diluted to low concentrations to lessen inflammation at the site of administration. The amount of liposomes administered will depend upon the particular camptothecin used, the disease state being treated and the judgment of the clinician, but will generally, in a human, be between about 0.01 and about 50 mg per kilogram of body weight, preferably between about 5 and about 40 mg/kg of body weight. Higher lipid doses are suitable for mice, for example, 50-120 mg/kg.

Suitable formulations for use in the present invention can be found, e.g., in Remington's Pharmaceutical Sciences, Mack Publishing Company, Philadelphia, Pa., 17^{th} Ed. (1985). Often, intravenous compositions will comprise a solution of the liposomes suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. These compositions can be sterilized by conventional sterilization techniques, such as filtration. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the composition may include lipid-protective agents, which protect lipids against free-radical and lipid-peroxidative damages on storage. Lipophilic free-radical quenchers, such as α.-tocopherol and water-soluble iron-specific chelators, such as ferrioxamine, are suitable. The concentration of liposomes in the carrier can vary. Generally, the concentration will be about 20-200 mg/mL. However, persons of skill can vary the concentration to optimize treatment with different liposome components or for particular patients. For example, the concentration may be increased to lower the fluid load associated with treatment.

The amount of camptothecin administered per dose is selected to be above the minimal therapeutic dose but below a toxic dose. The choice of amount per dose will depend on a number of factors, such as the medical history of the patient, the use of other therapies, and the nature of the disease. In addition, the amount of camptothecin administered may be adjusted throughout treatment, depending on the patient's response to treatment and the presence or severity of any treatment-associated side effects. In certain embodiments, the dosage of liposomal composition or the frequency of administration is approximately the same as the dosage and schedule of treatment with the corresponding free camptothecin. However, it is understood that the dosage may be higher or more frequently administered as compared to free drug treatment, particularly where the liposomal composition exhibits reduced toxicity. It is also understood that the dosage may be lower or less frequently administered as compared to free drug treatment, particularly where the liposomal composition exhibits increased efficacy as compared to the free drug. Exemplary dosages and treatment for a variety of chemotherapy compounds (free drug) are known and available to those skilled in the art and are described in, e.g., Physician's Cancer Chemotherapy Drug Manual, E. Chu and V. Devita (Jones and Bartlett, 2002).

In general, dosage for the camptothecin will depend on the administrating physician's opinion based on age, weight, and condition of the patient, and the treatment schedule. A recommended dose for free topotecan in Small Cell Lung Cancer is 1.5 mg/M² per dose, every day for 5 days, repeated every three weeks. Because of the improvements in treatment now demonstrated in the examples, below, doses of topotecan in liposomal topotecan in humans will be effective at ranges as low as from 0.015 mg/M²/dose and will still be tolerable at doses as high as 15 to 75 mg/M²/dose, depending on dose scheduling. Doses may be single doses or they may be administered repeatedly every 4 h, 6 h, or 12 h or every 1 d, 2 d, 3 d, 4 d, 5 d, 6 d, 7 d, 8 d, 9 d, 10 d or combination thereof. In particular embodiments, scheduling may employ a cycle of treatment that is repeated every week, two weeks, three weeks, four weeks, five weeks or six weeks or combination thereof. In one preferred embodiment, treatment is given once a week, with the dose typically being less than 1.5 mg/M². In another embodiment, the interval regime is at least once a week. In another embodiment, interval regime is at least once every two week, or alternatively, at least once every three weeks.

3. Combination Therapies

In numerous embodiments, liposomal compositions of the invention will be administered in combination with one or more additional compounds or therapies, such as surgery, radiation treatment, chemotherapy, or other camptothecins, including any of those described above. Liposomal compositions may be administered in combination with a second camptothecin for a variety of reasons, including increased efficacy or to reduce undesirable side effects. The liposomal composition may be administered prior to, subsequent to, or simultaneously with the additional treatment. Furthermore, where a liposomal composition of the present invention (which comprises a first camptothecin) is administered in combination with a second camptothecin, the second camptothecin may be administered as a free drug, as an independent liposomal formulation, or as a component of the liposomal composition comprising the first drug. In certain embodiments, multiple camptothecins are loaded into the same liposomes. In other embodiments, liposomal compositions comprising an camptothecin are formed individually and subsequently combined with other compounds for a single co-administration. Alternatively, certain therapies are administered sequentially in a predetermined order, such as in CHOP. Accordingly, liposomal compositions of the present invention may comprise one or more camptothecins.

Liposomal compositions of the invention, including, e.g., liposome-encapsulated camptothecins, can also be combined with anti-tumor agents such as monoclonal antibodies including, but not limited to, Oncolym™ (Techniclone Corp. Tustin, Calif.) or Rituxan™ (IDEC Pharmaceuticals), Bexxar™ (Coulter Pharmaceuticals, Palo Alto, Calif.), or IDEC-Y2B8 (IDEC Pharmaceuticals Corporation).

Other combination therapies known to those of skill in the art can be used in conjunction with the methods of the present invention. Examples of drugs used in combination with conjugates and other chemocamptothecins to combat undesirable side effects of cancer or chemotherapy include zoledronic acid (Zometa) for prevention of bone metastasis and treatment of high calcium levels, Peg-Filgrastim for treatment of low white blood count, SDZ PSC 833 to inhibit multidrug resistance, and NESP for treatment of anemia.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

### Example 1

### Influence of Temperature and Topotecan Concentration on Crystalline Particulate Formation in Liposomal Topotecan

Liposomal topotecan was prepared using MgSO₄ as described below. Essentially, liposomes comprising sphingomyelin and cholesterol (ESM/CH, 55:45 mol ratio) were prepared by hydration of a ethanol solution of ESM/CH in 300 mM MgSO₄ plus 200 mM sucrose. The resulting large multilamellar vesicles were size reduced by extrusion through 80 nm polycarbonate filters resulting in large unilamellar vesicles of mean diameter approximately 110-125 nm. Ethanol was removed by dialysis against the aqueous media used for hydration. The liposomes were then loaded with topotecan using a standard ionophore-mediated loading protocol as described previously (see, U.S. patent application Ser. No. 11/131,436). Following loading, the liposomal topotecan formulation was dialyzed against 10 volumes of 300 mM sucrose, 10 mM phosphate, pH 6 buffer followed by 10 volumes of 300 mM sucrose. Citrate buffer (pH 6.0) or phosphate buffer (pH 6.0) where then added to a final concentration of 10 mM. The final drug to lipid ratio of the preparation was 0.094 (wt/wt) and had a vesicle size of 110±40 nm diameter as measured by quasi-elastic light scattering.

The liposomal topotecan formulations were incubated at 5, 25, and 35° C., and topotecan crystal particulate formation was monitored over for six weeks.

Topotecan crystal particulates were counted using a high throughput assay. The assay employed a hemocytometer, which is a glass slide normally used in combination with a microscope for determining cell concentrations, such as in blood samples. It consists of a 0.1 mm deep chamber over which a glass cover slip is placed and the sample loaded by capillary action between the two surfaces. The surface of the hemocytometer chamber is marked by four 1 mm by 1 mm squares. Each 1 mm by 1 mm square is further scored into 16 squares. As the depth of the chamber is 0.1 mm, the volume contain beneath the 1 mm by 1 mm surface is 0.1 mm³ or 0.1 μl. For this study, four chamber surfaces, each containing four 1 mm by 1 mm surfaces (0.4 μL) were counted. The data are expressed as the average of the four 0.4 μl counts, ±one standard deviation. Thus if one particle were seen in the 0.4 μl volume, the number of particles calculated per ml would be 625. Typically, 0 to 3 particles (0-2000 particles per ml) were seen at time 0 and thus the estimated background count or limit of detection (LOD) is ˜2000 crystals/ml. The data were tabulated as particulates longer than 25 μm and the total number of particulates seen. The results generally correlated with those obtained using a USP-like filter-based particulate test method. Where the filter assay detected numerous crystals (well above USP limits), the hemocytometer also measured high crystal counts. Similarly, when the filter method detected particles on the order of 1000/ml or less, the hemocytometer counts were at or below the LOD for the hemocytometer assay.

At weekly time points, vials were taken for particulate analysis using a Hausser Scientific hemocytometer (VWR cat #15170-168) coated with rhodium to improve particle contrast. A 25-gauge needle was used to withdraw the liposomal topotecan formulations from the vials to apply to the hemocytometer. The particles and counting chamber of the hemocytometer were visualized using a Nikon Eclipse TE300 microscope fitted with a 10 or 20× objective lens. A fresh vial was used at each time point.

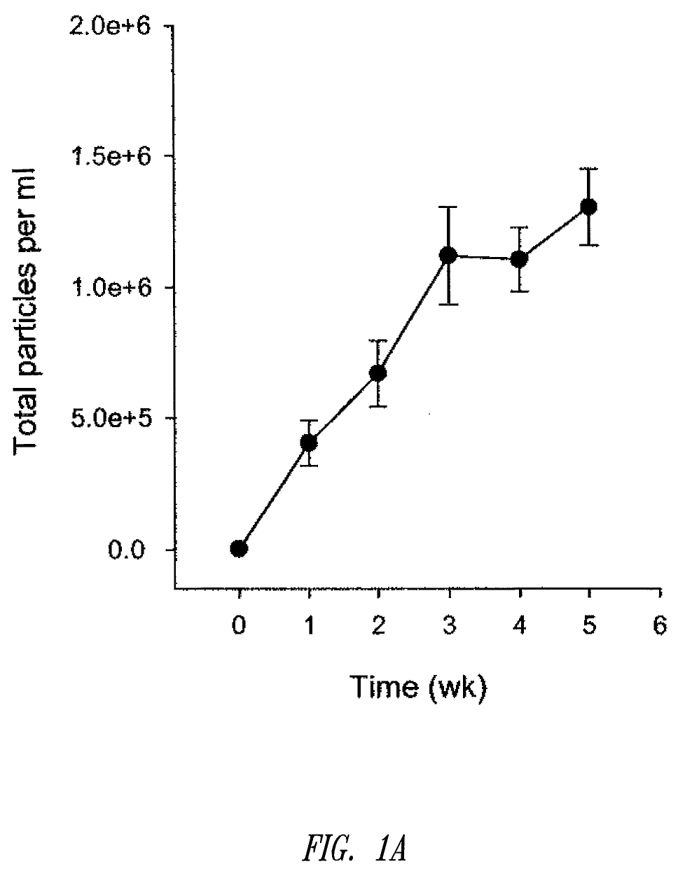

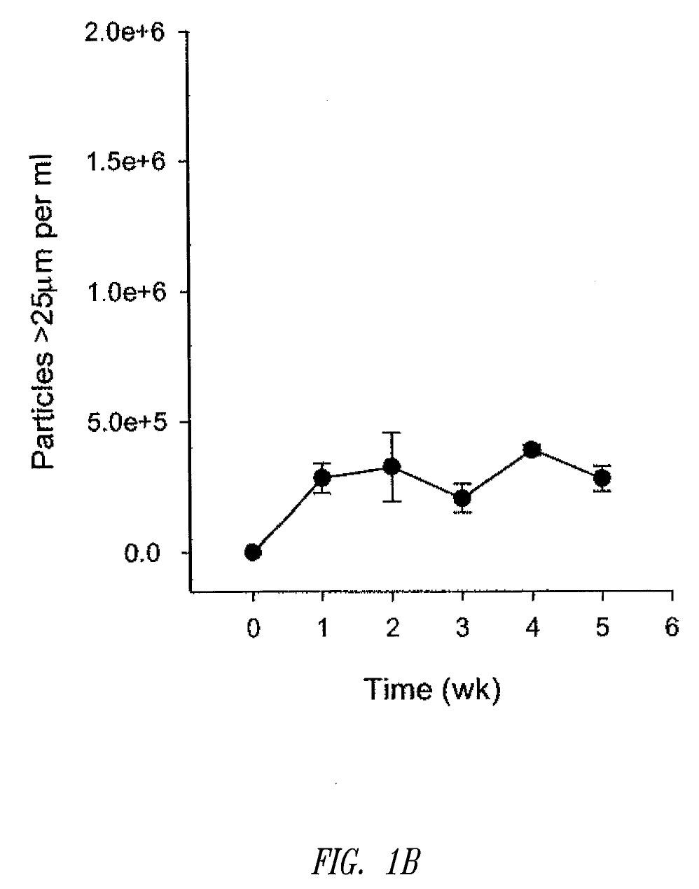

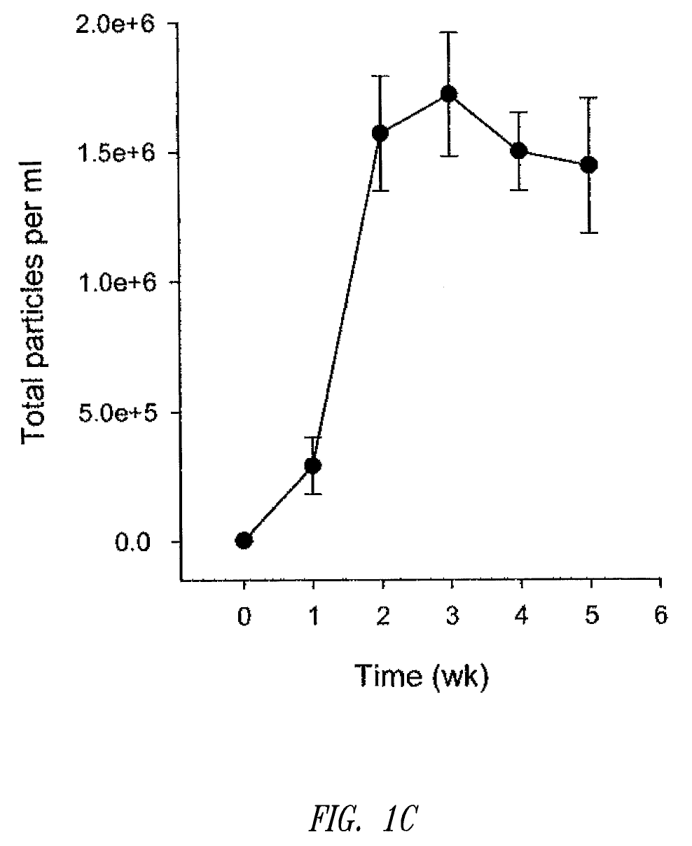

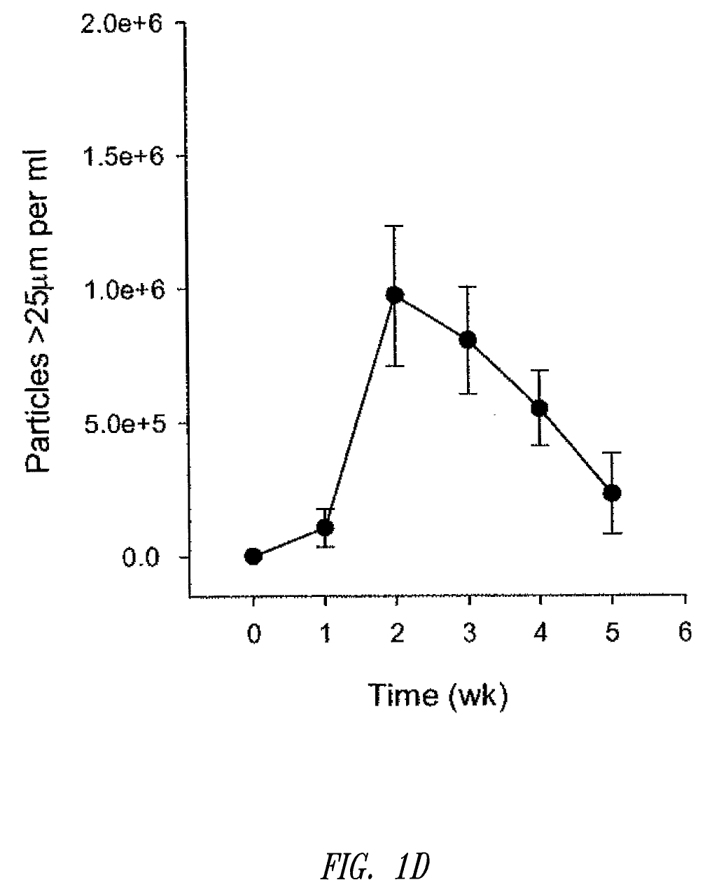

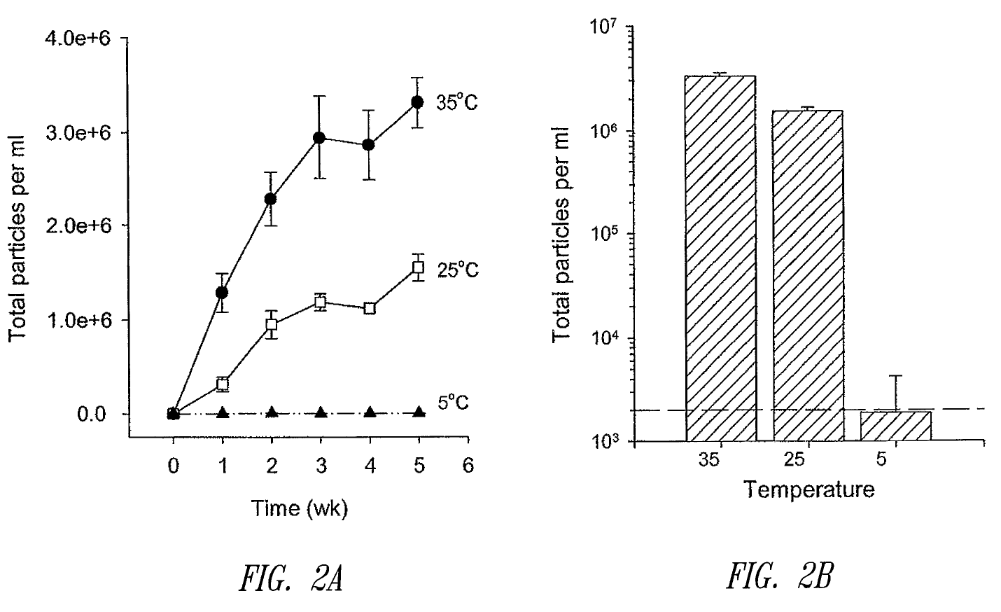

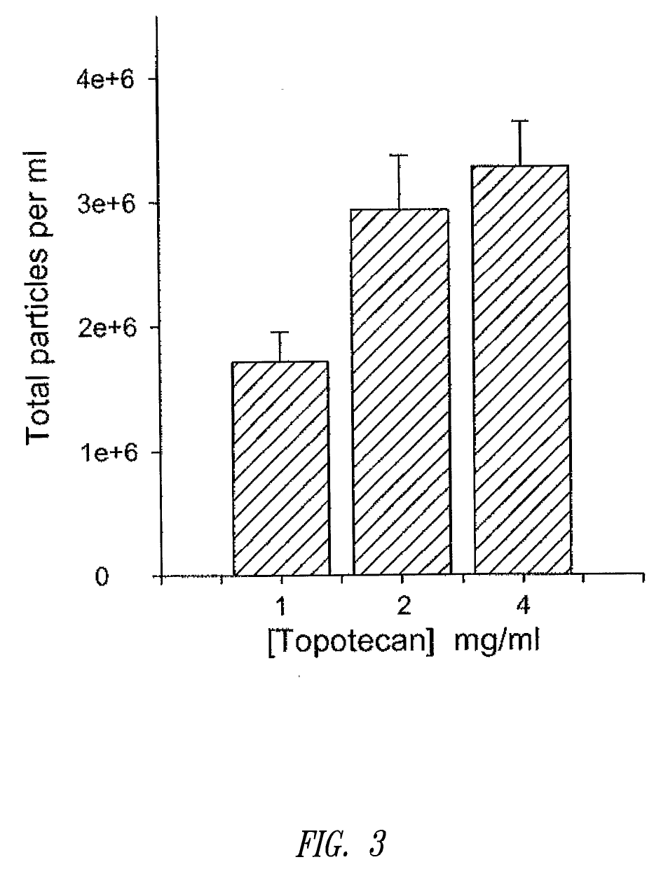

As shown in FIG. 1, rapid crystal formation was seen for the liposomal topotecan formulations described above on incubation at 35° C. Total crystal numbers per vial increased over six weeks in both formulations (i.e., with citrate buffer pH 6.0 or phosphate buffer pH 6.0). The initial rate of crystal formation appeared faster for the formulation in phosphate buffer (c.f. FIGS. 1A and 1C). Large crystals (>25 micron) were also observed in the formulations but the numbers of these large crystals appeared to plateau over the 5-week period in which they were separately counted. The kinetic of crystal formation were temperature dependent with faster development at 35° C. compared to 25° C. (FIG. 2). The magnitude of the temperature effect at 5 weeks is better observed in a semi-logarithmic plot (FIG. 2B). The concentration of liposomal topotecan in the vials was also varied (1, 2 and 4 mg/mL) and the influence on crystal formation determined (FIG. 3). Vials containing 4 mg/mL topotecan or 2 mg/mL topotecan had similar numbers of crystals at 3 week incubation at 35° C. Approximately two-fold fewer crystals were observed at 1 mg/mL topotecan concentration.

### Example 2

### Alternate External Buffers and Reduced pH Exhibit Reduced Liposomal Topotecan Crystal Formation

In order to determine the effect of pH and external buffer composition on liposomal topotecan stability and crystal formation, liposomal topotecan formulations were prepared as described in Example 1 using 300 mM MgSO₄, 200 mM sucrose as the internal solution. Following topotecan loading as described in Example 1, samples were prepared with external solutions comprising citrate, tartrate or phosphate buffers over a range of pH values and topotecan concentrations (Table 1). The formulations were then aliquoted (1 ml) into glass 2 ml vials, sealed, and incubated at 5, 25, or 35° C. Topotecan crystal particular formation was monitored as described for Example 1 for eight weeks.

TABLE 1
Sample Matrix Characterizing Different External
Buffers, pH and Topotecan Concentration.

| | | Sample ID | | |
|---|---|---|---|---|
| | pH | 1 mg/ml | 2 mg/ml | 4 mg/ml |
| Citrate | 6.0 | 1 | 5 | 9 |
| | 4.5 | 2 | 6 | 10 |
| | 4.0 | 3 | 7 | 11 |
| | 3.5 | 4 | 8 | 12 |
| Tartrate | 4.5 | 13 | 16 | 19 |
| | 4.0 | 14 | 17 | 20 |
| | 3.5 | 15 | 18 | 21 |
| Phosphate | 6.0 | 22 | 24 | 26 |
| | 3.5 | 23 | 25 | 27 |

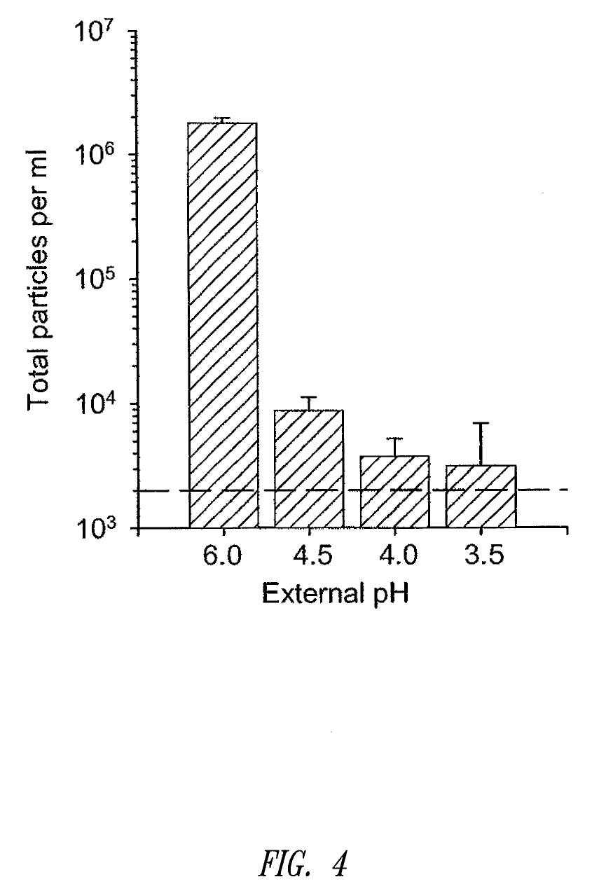

The effect of various external pHs was determined. Samples of liposomal topotecan (2 mg/ml) were incubated at 35° C. for five weeks in an external buffer of 300 mM sucrose, 10 mM citrate and pH range of 3.5 to 6.0. Remarkably, a 500-fold decrease in crystal particulates was observed when the external pH was lowered from pH 6 to pH 4.5 or below (FIG. 4).

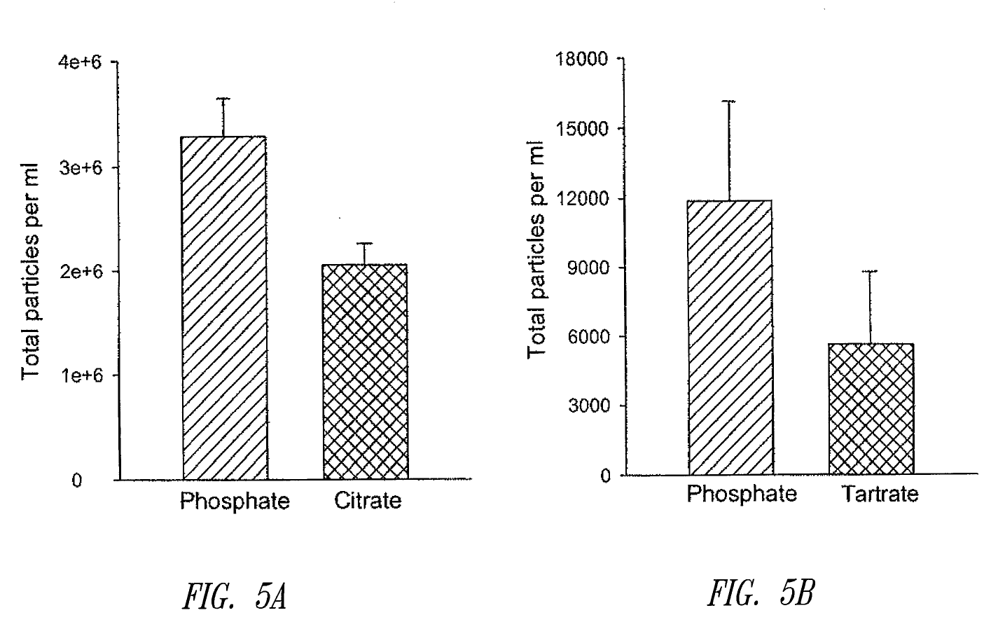

The comparative effect of using phosphate, citrate or tartrate buffers on the rate of crystal formation was also examined. Phosphate and citrate were compared at pH 6.0 because they have pKas of 7.2 and 5.4 respectively and hence are effective buffers at pH 6.0. In contrast phosphate and tartrate were compared at pH 4.0 as tartrate has a pKa of 3.2 and phosphate has a second pKa of 2.1. Phosphate buffer was found to promote the formation of approximately 2-fold more crystals compared to either citrate (FIG. 5A) or tartrate (FIG. 5B).

### Example 3

### Empty Liposomes Reduce Liposomal Topotecan Crystal Formation

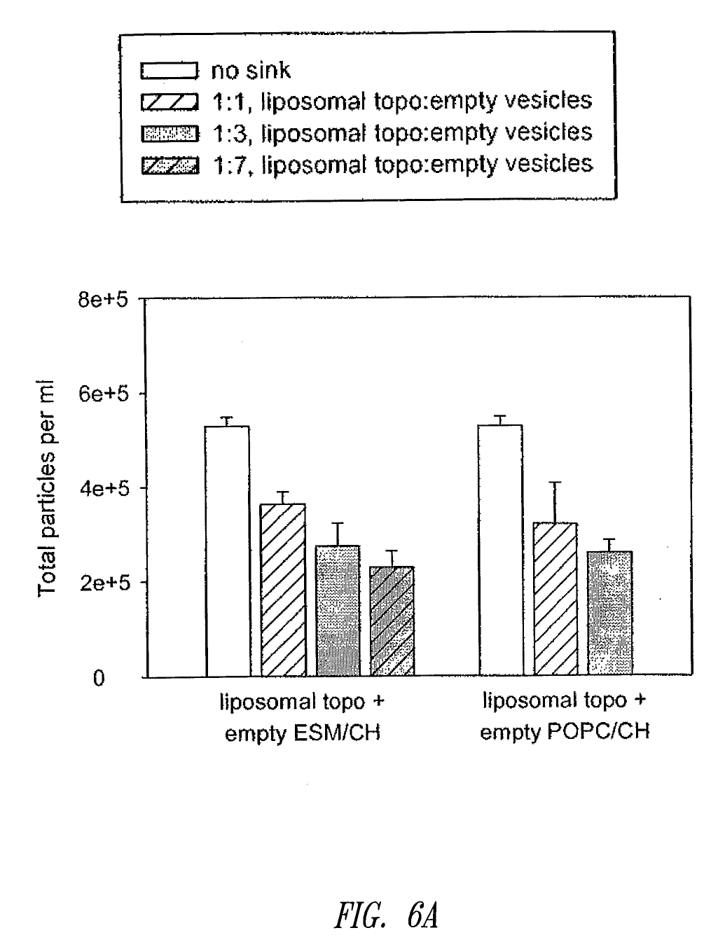

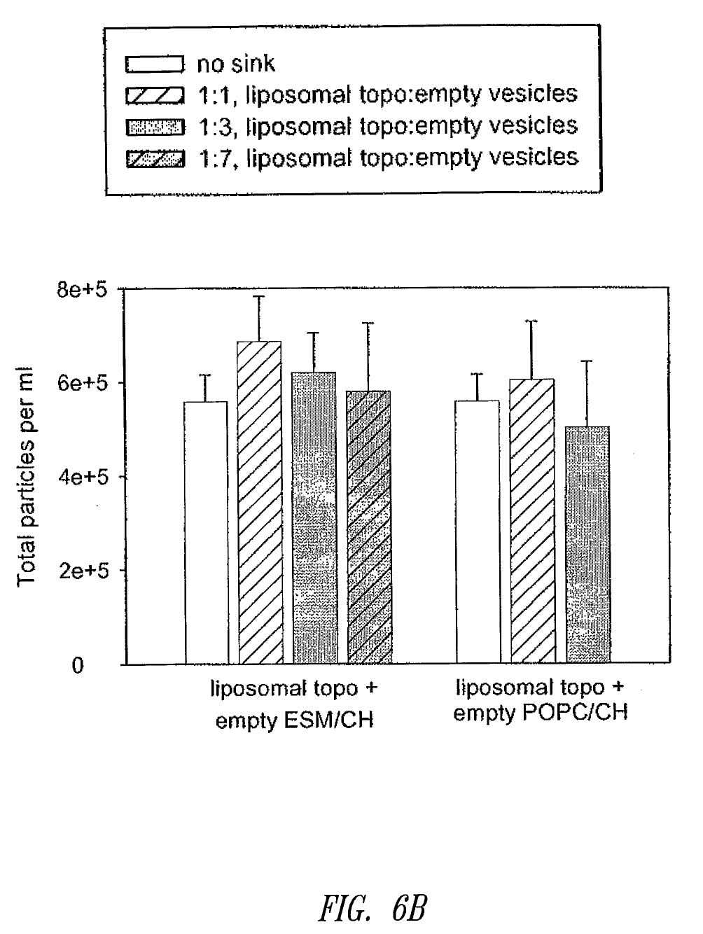

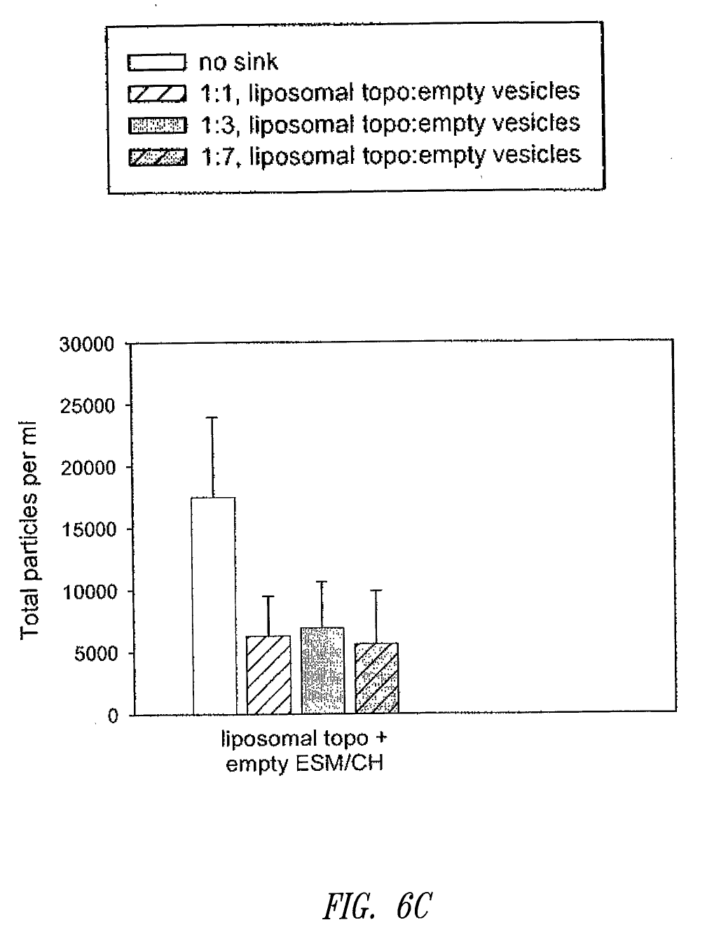

The effect of the addition of empty liposomes on topotecan stability and crystal formation was determined using liposomal topotecan formulations comprising MgSO₄ as described above. Empty vesicles consisting of 1-palmitoyl-2-oleoyl-glycero-3-phosphocholine:cholesterol (POPC:CH, 55:45 mol ratio) or (ESM/CH, 55:45 mol ratio) were added from a stock concentration of 50 mg/ml lipid to liposomal topotecan (0.5 mg/ml topotecan) in a final external buffer of 300 mM sucrose, 10 mM citrate, pH 6.0. The empty liposomes exhibited mean diameters equivalent to the topotecan-containing ESM/CH liposomes. The ratios of empty vesicle to liposomal topotecan examined were 0:1, 1:1, 3:1 and 7:1 (lipid w/wt). The mixtures were vialed in 1 ml aliquots and incubated at 25 or 35° C. After one week at 35° C., a reduction in crystal numbers was seen that correlated with the amount of empty vesicles present (FIG. 6A). This effect was the same for ESM and POPC-containing vesicles, and at its maximum, resulted in a 2-fold reduction in crystals compared to the control sample.

After two weeks incubation at 35° C., the effect was lost, and all samples exhibited a similar number of crystals (FIG. 6B). However, at 25° C., all samples containing empty vesicles still showed a significant reduction in crystal numbers compared to the control sample (FIG. 6C).

### Example 4

### Anti-Oxidants Reduce Topotecan Crystal Formation

The effect of the addition of antioxidants to liposomal topotecan formulations on crystal formation was examined using the anti-oxidants, ascorbic acid and α-tocopherol. These compounds are also referred to as free radical scavengers.

The effect of the addition of ascorbic acid was determined by incubating liposomal topotecan formulations in an external buffer containing ascorbic acid (ascorbic acid). Specifically, SM/CH (55:45 mol ratio, initial internal Mg²⁺ solution) or DHSM/CH (55:45 mol ratio, initial internal Mn²⁺ solution) topotecan formulations (D/L ratio 0.1, wt/wt) were incubated at 37° C. in external buffers comprising 300 mM sucrose, 10 mM phosphate, pH 6, or 300 mM sucrose, 10 mM phosphate, 10 mM ascorbic acid, pH 6. Crystal particle formation was monitored using a hemocytometer as described for Example 1.

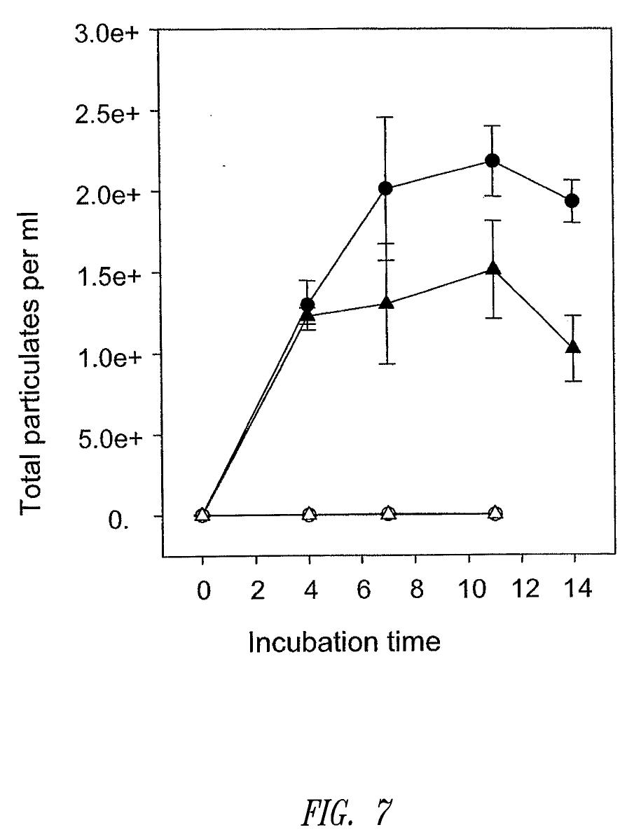

As shown in FIG. 7, liposomal topotecan formulations comprised of DHSM/CH containing Mn²⁺ as the internal cation show lower crystals levels compared to similar formulations comprising SM/CH and Mg²⁺ as the internal cation. Further, the presence of ascorbic acid in the external buffer dramatically decreased topotecan crystal formation. This effect was observed for both SM and DHSM liposomes and in the presence of both Mg²⁺ and Mn²⁺.

The effect of the addition of the anti-oxidant, α-tocopherol (alpha-tocopherol) was examined by solubilizing alpha-tocopherol in ethanol and incorporating it into the DHSM/CH lipid mixture at 0 to 2 mole percent during vesicle formation (as described above using 300 mM MnSO₄, 200 mM sucrose as the hydration buffer). The vesicles were then loaded with topotecan as described for Example 1, and incubated at 37° C. in an external buffer of 300 mM sucrose, 10 mM citrate, pH 6.

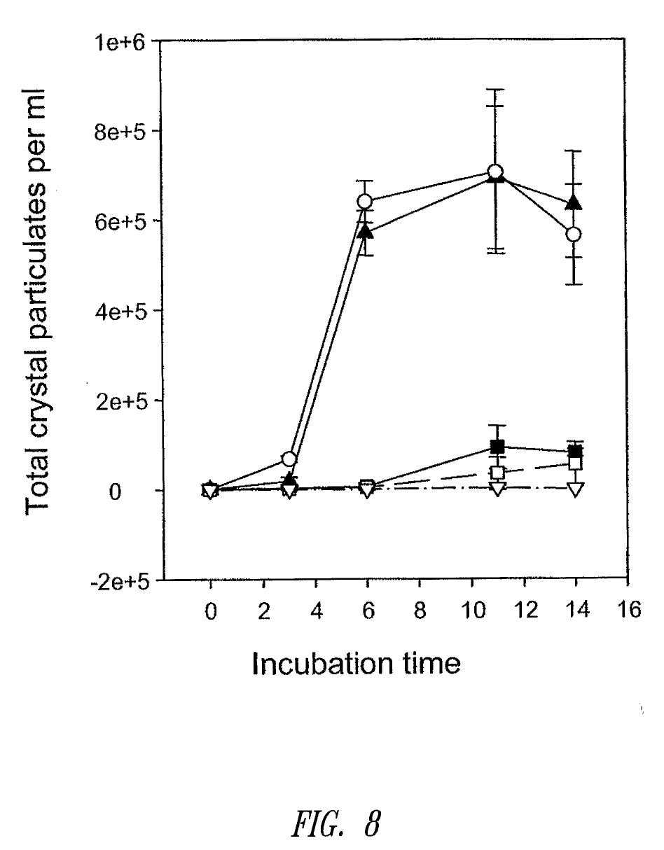

DHSM/CH (55:45 mol ratio) vesicles without alpha-tocopherol or with 0.2% alpha-tocopherol showed large increases in crystal formation by the six day time point (FIG. 8). However, vesicles with 0.5 to 2 mole percent alpha-tocopherol had much reduced crystal formation, and no crystals were detected in the 2% alpha-tocopherol sample over the 14 day time course examined (FIG. 8).

The vesicles were sized by quasi-elastic light scattering using a Nicomp particle sizer after the 14 day time point. An increase in vesicle size and distribution was observed for the 1 and 2 mol % alpha-tocopherol-containing samples, potentially indicating vesicle fusion and suggesting there is a limit in the amount of alpha-tocopherol that can be incorporated without affecting membrane stability.

These results demonstrate that anti-oxidants can be used to successfully reduce topotecan crystal formation. Accordingly, other anti-oxidants or free radical scavengers may also be used to reduce crystal formation. Other methods that would also reduce topotecan crystal formation include, but are not limited to, reducing oxygen content by purging the solutions with nitrogen and/or sealing the vialed liposomal topotecan under nitrogen.

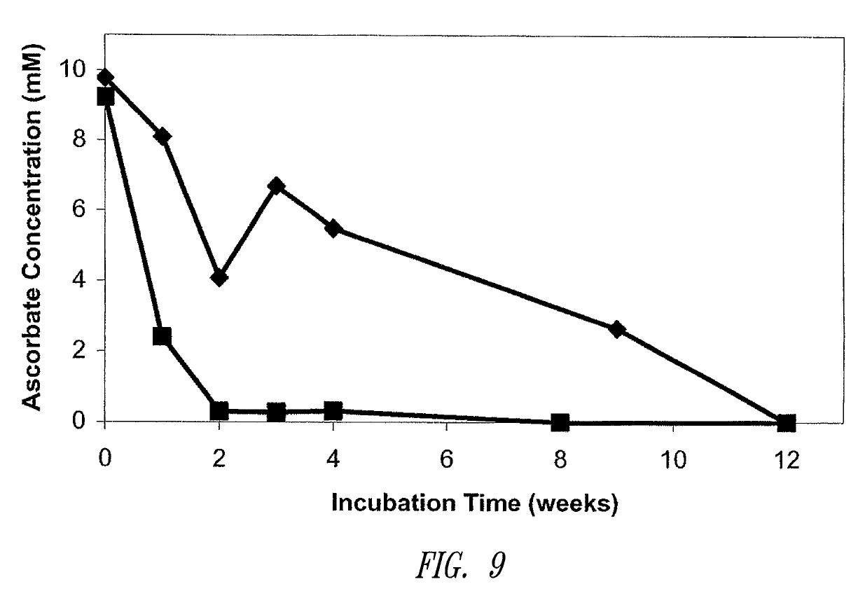

Analysis of ascorbic acid concentrations over time in liposomal topotecan samples showed a significant decrease in this antioxidant. A study was therefore conducted to determine if reducing the partial pressure of oxygen in liposomal topotecan formulations containing ascorbic acid (10 mM) could reduce the rate of loss. Liposomes composed of SM/CH (55:45) with MgSO₄ as the internal salt were prepared and loaded with topotecan as described for Example 1. The final external solution included ascorbic acid (10 mM). As shown in FIG. 9, purging of the liposomal topotecan formulation prior to vialling and vialling under nitrogen resulted in much slower ascorbic acid loss on subsequent incubation at 40° C. for up to 12 weeks. Accordingly inclusion of a process to reduce the partial pressure of oxygen in liposomal camptothecin formulation containing an antioxidant, such as ascorbic acid, is useful in reducing the rate of loss of the antioxidant and hence in ensuring that adequate concentrations of antioxidant are retained in the formulation to protect against camptothecin degradation.

### Example 5

### Influence of Lipid Composition, Internal Manganese and Ascorbic Acid on Particulate Formation in Liposomal Topotecan

A study was conducted to compare particulate formation in liposomal topotecan formulations composed of SM/CH and DHSM/CH (55:45) in the presence and absence of ascorbic acid. Liposomes were prepared and loaded with topotecan as described in Example 1. Liposomes composed of SM/CH were prepared comprising MgSO₄ or MnSO₄ in the internal solution. Liposomes composed of DHSM/CH were prepared comprising MnSO₄ in the internal solution. Formulations of both SM/CH and DHSM/CH liposomes were also prepared containing ascorbic acid (10 mM) in the external solution. These liposomal topotecan formulations are shown in Table 2.

TABLE 2
Liposomal topotecan formulations matrix.

| Formulation | | Internal | Ascorbic | |
|---|---|---|---|---|
| Name | Lipid | cation | acid | pH |
| SM/CH/Mg | SM | MgSO4 | No | pH 4 |
| SM/CH/Mg/AA | SM | MgSO4 | Yes | pH 4 |
| SM/CH/Mn/AA | SM | MnSO4 | Yes | pH 4 |
| DHSM/CH/Mn | DHSM | MnSO4 | No | pH 4 |
| DHSM/CH/Mn/AA | DHSM | MnSO4 | Yes | pH 4 |

These formulations were vialed and incubated at 5, 25 and 40° C. for up to 3 months. At 1, 2 and 3 months particulate counts were obtained using an in-house particle counting method employing approximately 1 mL of sample (Table 3). In addition, at 3 months particulate counts were conducted using the official USP particle count method (filtration method) (Table 4).

TABLE 3
Particulate crystal counts in liposomal topotecan formulations

| | | 1 month | | | 2 month | | | 3 month | | |
|---|---|---|---|---|---|---|---|---|---|---|
| Formulation | T = 0 | 5C | 25C | 40C | 5C | 25C | 40C | 5C | 25C | 40C |
| SM/CH/Mn/AA | no | — | no | no | no | no | no | no | no | 74* |
| SM/CH/Mg | no | — | no | 24698 | no | 308 | 42083 | no | 1368 | TNTC¹ |
| SM/CH/Mg/AA | no | — | no | no | no | no | 17 | no | no | 6** |
| DHSM/CH/Mn | no | — | no | no | no | no | no | no | no | no |
| DHSM/CH/Mn/AA | no | — | no | no | no | no | 1260 | no | no | 342* |

¹Too numerous to count
*Not typical topotecan crystals
**Only represents one actual observation

Liposomal topotecan formulated with MgSO₄ as the internal cation and without ascorbic acid shows significant number of crystals at 1 month at 40°. Further at 2 months crystal counts likely exceeding USP limits are seen both at 25 and 40° C. In contrast the same liposome formulation and internal cation including ascorbic acid shows none, or very low, crystal counts even at 3 months at 40° C. Similarly when SM/CH liposomes are loaded with topotecan using MnSO₄ and ascorbic acid included in the external solution, none, or very low, crystal counts are seen up to 3 months at 40° C. It should be noted that atypical crystals were seen in this formulation at 3 months at 40° C. and may not result from topotecan degradation products. Liposomal topotecan formulated in DHSM/CH liposomes using MnSO₄ and containing ascorbic acid show no crystals at 5 and 25° C. for up to 3 months. Crystals seen in this formulation at 40° C. are atypical and may not result from topotecan degradation. The same DHSM/CH formulation but without ascorbic acid surprisingly shows no crystals up to 3 months at any temperature. These in-house data were supported by analysis of the same formulations at 3 months using the USP method for determination of particle counts (Table 4).

TABLE 4
USP Particulate counts in liposomal topotecan formulations at 3 months.

| | Temp | Particles (excluding crystals) | | Topo Crystals | | Total Particulates | |
|---|---|---|---|---|---|---|---|
| | (C.) | >=10 um | >=25 um | >=10 um | >=25 um | >=10 um | >=25 um |
| SM/CH/Mn/AA | 5 | 14 | 11 | 0 | 0 | 14 | 11 |
| SM/CH/Mg | 5 | 16 | 6 | 0 | 0 | 16 | 6 |
| SM/CH/Mg/AA | 5 | 21 | 5 | 0 | 0 | 21 | 5 |
| DHSM/CH/Mn | 5 | 2 | 1 | 0 | 0 | 2 | 1 |
| DHSM/CH/Mn/AA | 5 | 13 | 6 | 0 | 0 | 13 | 6 |
| SM/CH/Mn/AA | 25 | 6 | 2 | 0 | 0 | 6 | 2 |
| SM/CH/Mg | 25 | 8 | 2 | 85 | 15 | 93 | 17 |
| SM/CH/Mg/AA | 25 | 6 | 4 | 0 | 0 | 6 | 4 |
| DHSM/CH/Mn | 25 | 7 | 3 | 0 | 0 | 7 | 3 |
| DHSM/CH/Mn/AA | 25 | 3 | 2 | 0 | 0 | 3 | 2 |
| SM/CH/Mn/AA | 40 | 12 | 3 | 0 | 0 | 12 | 3 |
| SM/CH/Mg | 40 | 3 | 2 | 6068 | 832 | 6071 | 834 |
| SM/CH/Mg/AA | 40 | 7 | 6 | 0 | 0 | 7 | 6 |
| DHSM/CH/Mn | 40 | 4 | 3 | 1 | 0 | 5 | 3 |
| DHSM/CH/Mn/AA | 40 | 8 | 2 | 0 | 0 | 8 | 2 |

The results obtained using the USP method confirms crystal counts obtained internally. The SM/CH topotecan formulation loaded using MgSO₄ without ascorbic acid in the external solution shows high particle counts at both 25 and 40° C. and these high particle counts arises almost exclusively from topotecan related crystals. This same formulation but containing ascorbic acid shows low particle counts (well within USP limits) at all temperatures. Similarly liposomal topotecan formulations comprising MnSO₄ in the internal solution show low particle counts at all temperatures. Finally, liposomal topotecan formulations comprising of DHSM/CH liposomes either in the presence or absence of ascorbic acid show low particulate counts. These results show that addition of an antioxidant, ascorbic acid, to liposomal topotecan formulations greatly reduces drug degradation and crystal formation. In addition, liposomes comprised of DHSM/CH are shown to greatly reduce crystal formation either in the presence or absence of ascorbic acid.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A liposomal camptothecin formulation adapted for increased camptothecin stability, comprising:
(a) a camptothecin encapsulated in a liposome;
(b) a first solution exterior of said liposome wherein said first solution has a pH less than or equal to 4.5; and
(c) a second solution interior of said liposome.

2. The formulation of claim 1, wherein said second solution comprises MnSO₄.

3. The formulation of claim 1, wherein said liposome comprises dihydrosphingomyelin and cholesterol.

4. The formulation of claim 1, wherein said formulation further comprises an anti-oxidant or free radical scavenger.

5. The formulation of claim 1 wherein said formulation further comprises empty liposomes.

6. The formulation of claim 1, wherein said first solution comprises a citrate or tartrate buffer.

7. The formulation of claim 1, wherein said liposome comprises dihydrosphingomyelin and cholesterol, wherein said formulation further comprises an anti-oxidant or free radical scavenger, and wherein said second solution comprises MnSO₄.

8. A liposomal camptothecin formulation adapted for increased camptothecin stability, comprising:
(a) a camptothecin encapsulated in a liposome;
(b) a first solution exterior of said liposome;
(c) a second solution interior of said liposome; and
(d) an anti-oxidant or free radical scavenger.

9. The formulation of claim 8, wherein said first solution has a pH less than or equal to 4.5.

10. The formulation of claim 8, wherein said second solution comprises MnSO₄.

11. The formulation of claim 8, wherein said liposome comprises dihydrosphingomyelin and cholesterol.

12. (canceled)

13. (canceled)

14. The formulation of claim 8, wherein said formulation further comprises empty liposomes.

15. The formulation of claim 8, wherein said first solution comprises a citrate or tartrate buffer.

16. The formulation of claim 4 or 8, wherein said anti-oxidant or free radical scavenger is ascorbic acid.

17. The formulation of claim 16, wherein the ascorbic acid is present at a concentration in the range of 1 mM to 100 mM.

18. The formulation of claim 17, wherein the concentration of the ascorbic acid is approximately 10 mM.

19. The formulation of claim 4 or 8, wherein said anti-oxidant or free radical scavenger is alpha-tocopherol.

20. The formulation of claim 19, wherein said alpha-tocopherol is present at a concentration in the range of 0.1 to 10 mole percent.

21. The formulation of claim 20, wherein said alpha-tocopherol is present at a concentration in the range of 0.4 to 3 mole percent.

22. The formulation of claim 21, wherein said alpha-tocopherol is present at a concentration of approximately 2 mole percent.

23. The formulation of claim 1 or 8, wherein said camptothecin is topotecan.

24. The formulation of claim 23, wherein said formulation is a unit dosage form of topotecan.

25. The formulation of claim 24, wherein said topotecan is present at a unit dosage form of about 0.01 mg/M²/dose to about 7.5 mg/M²/dose.

26.-32. (canceled)

33. A pharmaceutical composition adapted for intravenous administration of a liposome-encapsulated camptothecin, wherein said pharmaceutical composition comprises a formulation of claim 1 or 8.

34. (canceled)

35. The formulation or pharmaceutical composition of claim 1 or 8, wherein the first solution has a reduced oxygen content, as compared to the oxygen content under atmospheric conditions.

36. A method for reducing the accumulation of camptothecin degradation products in a solution containing a camptothecin encapsulated in a liposome, comprising
formulating a camptothecin encapsulated in a liposome with a solution exterior of the liposome with a pH less than or equal to 4.5, and a solution interior of said liposome having the pH of the solution exterior of said liposomes at or below 4.5.

37. The method of claim 36, wherein said interior solution comprises MnSO₄.

38. The method of claim 36, wherein said liposome comprises dihydrosphingomyelin and cholesterol.

39. The method of claim 36, wherein said solution or liposome further comprises an anti-oxidant.

40. The method of claim 36, further comprising empty liposomes in the solution.

41.-47. (canceled)

48. A kit comprising a liposome-encapsulated camptothecin for administration to a patient in need thereof, comprising:
(a) a vial comprising a solution containing a camptothecin encapsulated in a liposome, wherein said solution exterior of said liposome has a pH less than or equal to 4.5; and
(b) instructions for preparing and/or administering the liposome encapsulated camptothecin to a patient.

49. The kit of claim 48, wherein the interior of said liposome comprises MnSO₄.

50. The kit of claim 48, wherein said solution or liposome comprises an antioxidant.

51. The kit of claim 48, wherein the solution containing a camptothecin encapsulated in a liposome further contains empty liposomes.

52.-62. (canceled)

63. A method of treating a cancer, comprising administering the pharmaceutical composition of claim 33 to a patient in need thereof, such that said cancer is treated.


SEQUENCE LISTING (1 sequence)

SEQ ID NO: 1 - DNA, 4 bp, Artificial Sequence
  Description of Artificial Sequence Synthetic oligonucleotide
atta